(19) United States
(12) Patent Application Publication
(10) Pub. No.: US 2025/0051352 A1
(43) Pub. Date: Feb. 13, 2025

(54) DIARYLOXYBENZOHETERODIAZOLE COMPOUNDS DISUBSTITUTED WITH THIENOTHIOPHENE GROUPS

(71) Applicant: ENI S.P.A., Roma (IT)

(72) Inventors: Luigi ABBONDANZA, Novara (NO) (IT); Giuliana SCHIMPERNA, Novara (NO) (IT); Alessandra TACCA, Novara (NO) (IT); Rosamaria MARRAZZO, Novara (NO) (IT)

(21) Appl. No.: 18/718,332

(22) PCT Filed: Dec. 9, 2022
(86) PCT No.: PCT/IB2022/061969

§ 371 Date: Jun. 10, 2024

(30) Foreign Application Priority Data
Dec. 10, 2021 (IT) 102021000031064

(51) Int. Cl. C07D 495/04; H10K 10/46; H10K 30/20; H10K 30/57; H10K 30/85; H10K 85/10; H10K 85/60

(52) U.S. Cl. CPC C07D 495/04; H10K 85/113; H10K 85/657; H10K 85/6576; H10K 10/462; H10K 30/20; H10K 30/57; H10K 30/85

(57) ABSTRACT

Diaryloxybenzoheterodiazole compound disubstituted with thienothiophene groups having general formula (I):

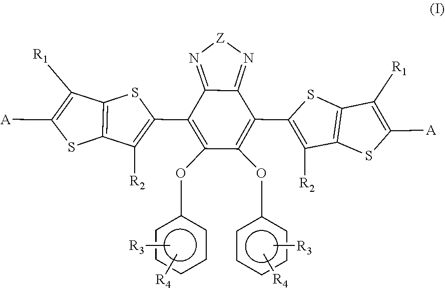

wherein:
   Z represents a sulfur atom, an oxygen atom, a selenium atom; or a NR₅ group wherein R₅ is selected from C₁-C₃₀;
   R₁ and R₂, identical to or different from each other,
   and R₃ and R₄, identical to or different from each other, represent a hydrogen atom;
   A represents an electron acceptor group having general formula (II):

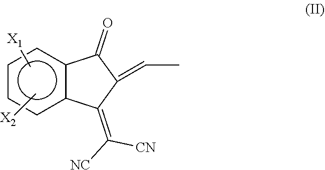

wherein X₁ and X₂, identical to or different from each other, represent a hydrogen atom, or a halogen atom; or are selected from C₁-C₃₀.

The compound is used as an electron acceptor compound in organic photovoltaic devices (or solar devices); is used in perovskite-based photovoltaic cells (or solar cells) in the layer based on electron transport material (“Electron Transport Layer”—ETL); and is used in the construction of organic thin film transistors, or of organic field effect transistors.

### CROSS-REFERENCE TO RELATED APPLICATIONS

This application is a 35 U.S.C. § 371 National Stage patent application of PCT/IB2022/061969 filed 9 Dec. 2022, which claims the benefit of Italian patent application 102021000031064, filed on 10 Dec. 2021, the disclosures of which are incorporated herein by reference in their entirety.

### TECHNICAL FIELD

The present disclosure relates to a diaryloxybenzoheterodiazole compound disubstituted with thienothiophene groups.

More particularly, the present disclosure relates to a diaryloxybenzoheterodiazole compound disubstituted with thienothiophene groups having general formula (I) reported below.

Said diaryloxybenzoheterodiazole compound disubstituted with thienothiophene groups having general formula (I) can be advantageously used as an electron acceptor compound in organic photovoltaic devices (or solar devices) selected, for example, from binary, ternary, quaternary, organic photovoltaic cells (or solar cells), having both simple and tandem architecture, organic photovoltaic modules (or solar modules), both on rigid support and on flexible support. Furthermore, said diaryloxybenzoheterodiazole compound disubstituted with thienothiophene groups having general formula (I) can be advantageously used in perovskite-based photovoltaic cells (or solar cells) in the layer based on electron transport material (“Electron Transport Layer”—ETL). Moreover, said diaryloxybenzoheterodiazole compound disubstituted with thienothiophene groups having general formula (I) can be advantageously used in the construction of organic thin film transistors (OTFTs), or of organic field effect transistors (OFETs).

The present disclosure also relates to an organic photovoltaic device (or solar device) selected, for example, from binary, ternary, quaternary, organic solar cells, having both simple and tandem architecture, organic photovoltaic modules (or solar modules), both on a rigid support and on a flexible support, comprising at least one diaryloxybenzoheterodiazole compound disubstituted with thienothiophene groups having general formula (I).

The present disclosure also relates to a perovskite-based photovoltaic cell (or solar cell) wherein the layer based on electron transport material (“Electron Transport Layer”—ETL) comprises at least one diaryloxybenzoheterodiazole compound disubstituted with thienothiophene groups having general formula (I).

The present disclosure also relates to organic thin film transistors (OTFTs), or to organic field effect transistors (OFETs) comprising at least one diaryloxybenzoheterodiazole compound disubstituted with thienothiophene groups having general formula (I).

### BACKGROUND

The market for photovoltaic cells (or solar cells) is currently dominated by crystalline silicon-based cells, due to their high efficiency and thanks to a well-established technology. Photovoltaic cells (or solar cells) can be divided into “generations” based on the characteristics of the photoactive material used in them. Thus, for example, we have:
first generation photovoltaic cells (or solar cells) based on crystalline silicon, commercially available;
second generation photovoltaic cells (or solar cells), such as, for example, photovoltaic cells (or solar cells) based on copper-indium and gallium (CIGS), or photovoltaic cells (or solar cells) based on cadmium telluride (CdTe), or photovoltaic cells (or solar cells) based on gallium arsenide (GaAs) and amorphous silicon, commercially available;
third generation photovoltaic cells (or solar cells) such as, for example, photovoltaic cells (or solar cells) based on copper zinc tin sulphide (CZTS), or perovskite-based photovoltaic cells (or solar cells), or dye sensitized photovoltaic cells (or solar cells) (DSSCs), or photovoltaic cells (or solar cells) based on quantum dots, or organic photovoltaic cells (or solar cells) (OPV) based on mixtures comprising electron acceptor compounds (A) which can be small molecules or polymers, and electron donor compounds (D) which can be small molecules or polymers, which are the photovoltaic cells (or solar cells) so-called emerging and the subject of continuous studies.

The success of one technology over another is influenced by many factors: for example, in the specific case of photovoltaic cells (or solar cells), their success will depend on their efficiency, lightness, cost-effectiveness, stability over time and also on their industrial scalability. In particular, their scalability at industrial level depends on further factors such as, for example, the abundance and toxicity of the raw materials used, the stability of these raw materials [for example, in some cases, photovoltaic cells (or solar cells) need an encapsulation in order to make them stable over time], the simplicity of the technology adopted for their production. An important role is also played by the environmental impact and the life cycle of these photovoltaic cells (or solar cells).

Despite the fact that organic photovoltaic cells (or solar cells) generally have lower efficiencies than first and second generation photovoltaic cells (or solar cells) [in recent years there has seen an increase in performance comparable to those of photovoltaic cells (or solar cells) belonging to the other more mature generations that have reached the plateau, and small-scale efficiencies of up to about 18% have been detected], they have enormous potential.

For example, among the many advantages of organic photovoltaic cells (or solar cells) can be mentioned: lightness, flexibility, semi-transparency, activation both in diffused light and by artificial light which makes them usable also in “indoor”, the ease and potential low cost of manufacturing using normal printing techniques such as, for example, the “roll-to-roll” (R2R) methodology reported, for example, by Valimaki M. et al, in “Nanoscale” (2015), Vol. 7, p. 9570-9580. Moreover, thanks to their flexibility and transparency, organic photovoltaic cells (or solar cells) can be easily integrated into buildings and windows and used in architectural design structures as reported, for example, by Burgués-Ceballos I. et al., in “Journal of Material Chemistry A” (2020), Vol. 8, p. 9882-9895.

The studies relating to organic photovoltaic cells (or solar cells) date back to about 40 years ago as reported, for example, by Tang C. W., in “Applied Physic Letters” (1986), Vol. 48, p. 183-185.

The elementary process of converting light into electric current in an organic photovoltaic cell (or solar cell) takes place through the following steps:
1. absorption of photons by the electron donor compound (D) or by the electron acceptor compound (A) [the absorption of each photon leads to the formation of an exciton, i.e. of a pair of charge carriers “electron-electronic gap (or hole);
2. diffusion of the exciton in a region of the compound that has absorbed the photon up to the interface with the compound that has not absorbed the photon [i.e. interface electron donor compound (D)-electron acceptor compound (A)], wherein dissociation of the exciton can occur;
3. dissociation of the exciton in the two charge carriers: electron (−) in the accepting phase [i.e. in the electron acceptor compound (A)] and electron gap (or hole) (+) in the donor phase (i.e. in the electron donor compound);
4. transport of the charges thus formed to the cathode [electron (−) through the electron acceptor compound (A)] and to the anode [electron gap (or hole) (+) through the electron donor compound (D)], with generation of an electric current in the circuit of the organic photovoltaic cell (or solar cell).

The photoabsorption process with exciton formation involves the excitation of an electron from the HOMO (E_{HOMO}) energy level (“Highest Occupied Molecular Orbital”) to the LUMO (E_{LUMO}) energy level (“Lowest Unoccupied Molecular Orbital”) of the compound that absorbed the photons. Subsequently, an electron is released from the LUMO energy level (E_{LUMO}) of the electron donor compound (D) to the LUMO energy level (E_{LUMO}) of the electron acceptor compound (A), or an electron is released from the HOMO energy level (E_{HOMO}) of the electron donor compound (D) to the HOMO energy level (E_{HOMO}) of the electron acceptor compound (A).

As mentioned above, in organic photovoltaic cells (or solar cells) the photoactive material is a mixture comprising electron acceptor compounds (A) and electron donor compounds (D). Both compounds absorb photons generating excitons and the photogenerated excitons from the electron acceptor compound (A) and from the electron donor compound (D) are separated by electron transfer and electron gap (or hole) transfer. Said electron transfer occurs spontaneously if the energy difference between the LUMO energy level (E_{LUMO}) of the electron donor compound (D) and the LUMO energy level (E_{LUMO}) of the electron acceptor compound (A) (ΔE_{LUMO, D-A}) is greater than zero and in any case greater than a threshold value that depends on the pair electron donor compound (D)—electron acceptor compound (A), and if the energy difference between the energy level HOMO (E_{HOMO}) of the electron donor compound (D) and the energy level HOMO (E_{HOMO}) of the electron acceptor compound (A) (ΔE_{HOMO, D-A}) is greater than zero and in any case higher than a threshold value that depends on the pair electron donor compound (D)—electron acceptor compound (A). To have an efficient dissociation of the exciton in the two charge carriers, the energy levels of the electron donor compound (D) and of the electron acceptor compound (A) must be aligned (i.e. correctly positioned). In order to absorb as much sunlight as possible, it would be better for electron acceptor compounds (A) and electron donor compounds (D) to absorb light of different wavelengths. Generally, a ΔE_{LUMO, D-A}>0.3 eV is required to have an efficient electron transfer while, as regards the transfer of electronic gaps (or holes), it has been observed that it can be efficient even if ΔE_{HOMO, D-A}<0.3 eV [said value must in any case be positive as reported for example, by Zhan C. et al., in “Journal Materials Chemistry A” (2018), Vol. 6, p. 15433-15455].

Another important feature of the compounds used in the production of organic photovoltaic cells (or solar cells) is the mobility of electrons in the electron acceptor compound (A) and of the electron gaps (or holes) in the electron donor compound (D), which determines the ease with which the electrical charges, once photogenerated, reach the electrodes.

Electron mobility, i.e. the mobility of electrons in the electron acceptor compound (A) and of the electron gaps (or holes) in the electron donor compound (D), in addition to being an intrinsic property of the compounds used, is also strongly influenced by the morphology of the photoactive layer, which in turn depends on the mutual miscibility of the compounds used in said photoactive layer and on their solubility. To this end, the phases of said photoactive layer must neither be too dispersed nor too segregated.

The morphology of the photoactive layer is also critical as regards the effectiveness of the dissociation of the photogenerated electron gap (hole)-electron pairs. In fact, the average life time of the exciton is such that it is able to diffuse into the organic material for an average distance which, for organic compounds, is generally around 10 nm-20 nm. Consequently, the phases of the electron donor compound (D) and of the electron acceptor compound (A) must be organized into nanodomains of comparable size with these diffusion lengths. Furthermore, the contact area (i.e. interface) between the electron donor compound (D) and the electron acceptor compound (A), must be as large as possible and there must be preferential paths to the electrical contacts. Furthermore, this morphology must be reproducible and must not change over time [see, for example, Gaitho F. M. et al, “Physical Sciences Reviews” (2018), doi: 10.1515/psr-2017-0102].

In the simplest way to operate, organic photovoltaic cells (or solar cells) are manufactured by introducing between two electrodes, usually made of indium tin oxide (ITO) (anode) and aluminum (Al) (cathode), a thin layer (about 100 nanometers) of a mixture of the electron acceptor compound (A) and the electron donor compound (D) [bulk heterojunction]. Generally, in order to create a layer of this type, a solution of the two components is prepared [i.e. electron acceptor compound (A) and electron donor compound (D)] and, subsequently, a photoactive layer is created on the anode [indium-tin oxide (ITO)] starting from said solution, using appropriate deposition techniques such as, for example, spin-coating, spray-coating, ink-jet printing, slot die coating, gravure printing, screen printing, and the like. Finally, the counter electrode [i.e. the aluminum cathode (Al)] is deposited on the dried photoactive layer by means of known techniques, for example, by evaporation. Optionally, between the anode and the photoactive layer and/or between the cathode and the photoactive layer, other additional layers can be introduced (called interlayers or buffer layers) capable of performing specific functions of an electrical, optical, or mechanical nature.

Generally, for example, in order to favor the reaching of the anode [indium-tin oxide (ITO)] by the electronic gaps (or holes) and at the same time of blocking the transport of the electrons, thus improving the collection of the positive charges by the anode and inhibiting the recombination phenomena, before creating the photoactive layer starting from the mixture of the electron acceptor compound (A) and the electron donor compound (D) as described above, a layer starting from an aqueous suspension comprising PEDOT:PSS [poly (3,4-ethylenedioxythiophene):sulphonated polystyrene], is deposited, using suitable deposition techniques such as, for example, spin-coating, spray-coating, ink-jet printing, slot die coating, gravure printing, screen printing, and the like.

In the state of the art, the electron donor compound (D) most commonly used in the production of organic photovoltaic cells (or solar cells) is the regioregular poly(3-hexylthiophene) (P3HT). This polymer has excellent electronic and optical characteristics [e.g., good values of energy levels HOMO (E_{HOMO}) and LUMO (E_{LUMO}), good molar absorption coefficient (s)], good solubility in the solvents that are used to manufacture the organic photovoltaic cells (or solar cells), and a fair mobility of electronic gaps (or holes).

Other examples of polymers which can be advantageously used as electron donor compounds (D) are: PCDTBT {poly[N-9″-heptadecanyl-2,7-carbazole-alt-5,5-(4′,7′-di-2-thienyl-2′,1′,3′-benzothiadiazole]}, the polymer PCPDTBT {poly[2,6-(4,4-bis-(2-ethylhexyl)-4H-cyclopenta[2,1-b;3,4-b′]-dithiophene)-alt-4,7-(2,1,3-benzothiadiazole)]}, the polymer PffBT4T-2OD {poly[(5,6-difluoro-2,1,3-benzothiadiazol-4,7-diyl)-alt-(3,3′″-di(2-octyldodecyl)-2,2′,5′,2″,5″,2′″-quaterthiophen-5,5′″-diyl)]}.

As for electron acceptor compounds (A), the history of organic photovoltaic cells (or solar cells) can be divided into two periods as reported, for example, by Zhan C. et al., in “Journal of Material Chemistry A” (2018), Vol. 6, p. 15433-15455:
the period of fullerenes wherein the electron acceptor compounds (A) were derivatives of fullerenes such as, for example, methyl ester of [6,6]-phenyl-C₆₁-butyric acid (PC61BM), methyl ester of acid (6,6)-phenyl-C₇₁-butyric acid (PC7TBM), still widely used today also in coupling with non-fullerene electron acceptor compounds (A) in ternary or quaternary organic photovoltaic cells (or solar cells);
the period of non-fullerenes wherein the electron acceptor compounds (A) were non-fullerene compounds as reported, for example, by Yan H. et al., in “Chemical Reviews” (2018), Vol. 118, 7, p. 3447-3507; Yang Y. et al., in “Nature Photonics” (2018), Vol. 12, p. 131-142; Gao F. et al., in “Nature Materials” (2018), Vol. 17, p. 119-128; McCulloch I. et al., In “Chemical Society Reviews” (2019), Vol. 48, p. 1596-1625, which led to an improvement in the efficiencies of organic photovoltaic cells (or solar cells) up to 18%, approaching the efficiencies of first and second generation photovoltaic cells (or solar cells) and thus increasing the interest in this type of technology.

The fullerene derivatives reported above offer certain advantages linked to their structure such as, for example, stability and efficient isotropic transport, due to the delocalization of the LUMO (E_{LUMO}) energy level over their entire surface. However, alongside these advantages, the derivatives of fullerene suffer from some intrinsic problems such as, for example:
weak absorption in the visible and near infrared regions (NIR);
low miscibility with most polymers;
high tendency to aggregate, which can create problems of long-term morphological stability;
values of energy levels HOMO (E_{HOMO}) and LUMO (E_{LUMO}) not very adjustable, since even the introduction of functional groups does not greatly alter the energy levels of said fullerene derivatives.

Compared to fullerene derivatives, non-fullerene compounds have significant advantages such as, for example:
adjustable band-gap values since, depending on the type of compound and its design, the non-fullerene compounds can absorb light in various areas of the solar spectrum and extend the absorption up to the near infrared (NIR);
adjustable values of energy levels HOMO (E_{HOMO}) and LUMO (E_{LUMO}) according to the structure of the compound;
adjustable planarity and crystallinity with greater control of the morphology of the active layer and a consequent increase in the stability of the photovoltaic cells (or solar cells) wherein they are used.

Non-fullerene electron acceptor compounds are, therefore, known in the art.

For example, Yao J. et al, in “Polymer Chemistry” (2013), Vol. 4, p. 4631-4638, report non-fullerene electron acceptor compounds comprising perylene diimides capable of providing organic photovoltaic cells (or solar cells) with efficiencies up to 1.95%.

Liu J. et al., in “Nature Energy” (2016), Vol. 1, Article No. 16089, p. 1-7, report non-fullerene electron acceptor compounds comprising naphthalene diimides capable of providing organic photovoltaic cells (or solar cells) with efficiencies up to 9.5%.

Lin Y. et al., in “Advanced Materials” (2015), Vol. 27, Issue 7, p. 1170-1174, report the design and synthesis of a new electron acceptor compound called ITIC based on a “core” comprising seven fused rings (indacenodithieno[3,2-b]thiophene, IT) substituted with four 4-hexyl-phenyl groups, and end-capped with 2-(3-oxo-2,3-dihydroinden-1-ylidene)malononitrile (INCN) groups capable of giving organic photovoltaic cells (or solar cells) with efficiencies up to at 6.8%.

Sauvè G., in “The Chemical Record” (2019), Vol. 19, p. 1078-1092, reports a series of electron acceptor compounds with different designs including a conjugated compound A-D-A with a planar structure wherein D is a rigid unit and has orthogonal side chains so as to control the aggregation capable of providing organic photovoltaic cells (or solar cells) with efficiencies up to 14%.

Yao H. et al., in “Nature Communications” (2019), Vol. 10, p. 10351-10355, report non-fullerene chlorinated electron acceptor compounds capable of giving organic photovoltaic cells (or solar cells) with efficiencies up to 16.5%.

Zou Y. et al., in “Joule” (2019), Vol. 3, p. 1140-1151, report a new class of non-fullerene electron acceptor compounds, having a central “core” based on fused rings (dithienothiophen[3.2-b]-pyrrolobenzothiadiazole) and benzothiadiazole capable of giving organic photovoltaic cells (or solar cells) with efficiencies up to 15.7%.

The above reported electron acceptor compounds having a long central “core” based on fused rings, while being able to give organic photovoltaic cells (or solar cells) having excellent performance, particularly in terms of efficiency, are very complicated molecules whose synthesis requires either many steps, or the use of very expensive starting materials and, consequently, their industrial development can be problematic.

Currently, non-fullerene electron acceptor compounds with a shorter central core are emerging.

For example, Chen H. et al, in “Journal of Material Chemistry A” (2018), Vol. 6, p. 12132-12141, report three non-fullerene electron acceptor compounds of the acceptor-donor-core-donor-acceptor (A-D-C-D-A) type which have the same electron donor part (D) and the same terminal electron acceptor part (A) but a different core (C). Among said electron acceptor compounds, the one having a 2,5-difluorobenzene core is capable of giving organic photovoltaic cells (or solar cells) with the highest efficiency equal to 10.97%.

Bo Z. et al, in “Nature Communications” (2019), Vol. 10, p. 3038-3047, report non-fullerene electron acceptor compounds having a core of non-covalent fused rings terminated with two dicyanoindanone molecules capable of giving organic photovoltaic cells (or solar cells) with efficiencies up to 13.24%.

In order to capture a greater amount of light, ternary organic photovoltaic cells (or solar cells) are also of considerable interest as reported, for example, by Chang L. et al., in “Organic Electronics” (2021), Vol. 90, 106063, wherein the photoactive layer consists of three compounds, having complementary absorption spectra. Unlike binary photovoltaic cells (or solar cells), ternary photovoltaic cells (or solar cells) contain a third compound (which can be both donor and acceptor), which can be used in smaller quantities than the other two. There are various principles useful for the selection of the third compound such as, for example, (1) complementary absorption to the spectrum of the original binary mixture, in order to absorb the greatest number of photons; (2) appropriate values of the HOMO and LUMO energy levels, i.e. they must be arranged in cascade as seen for binary photovoltaic cells (or solar cells) so that the excitons can be effectively separated; (3) good compatibility in order to improve the morphology of the photoactive layer (as reported, for example, by Yang C. et al., In “Organic Electronics” (2021), Vol. 91, 106085). The addition of said third compound can lead to an improvement in the performance of the organic photovoltaic cells (or solar cells) and to greater stability. The organic photovoltaic cells (or solar cells) comprising non-fullerene electron acceptor compounds, can be of three types: D-FA-NFA, D-NFA-NFA, DD-NFA, wherein D=electron donor compound, FA=fullerene electron acceptor compound, NFA=non-fullerene electron acceptor compound).

Examples of quaternary photovoltaic cells (or solar cells) are reported, for example, by Bi Z. et al., in “Advanced Functional Materials” (2019), Vol. 29, 1806804; or by Vincent P. et al., in “Energies” (2019), Vol. 12, 1838; or by Li W. et al, in “Macromolecular Rapid Communications” (2019), Vol. 40, Issue 21, 190353.

Since organic photovoltaic devices (or solar devices), in particular the so-called emerging organic photovoltaic cells (or solar cells), are the subject of continuous research and studies, the study of new non-fullerene electron acceptor compounds having simple structures and easy synthetic preparation, it is still of great interest.

### SUMMARY

The Applicant therefore posed the problem of finding new non-fullerene electron acceptor compounds having a simple structure and easily synthesized, capable of being advantageously used in organic photovoltaic devices (or solar devices), in particular in binary, ternary, quaternary, organic photovoltaic cells (or solar cells), having both simple and tandem architecture.

The Applicant has now found that the diaryloxybenzoheterodiazole compounds disubstituted with thienothiophene groups having the specific general formula (I) reported below can be advantageously used as non-fullerene electron acceptors in organic photovoltaic devices (or solar devices), in particular in organic polymer photovoltaics cells (or solar cells). Furthermore, said diaryloxybenzoheterodiazole compounds disubstituted with thienothiophene groups having general formula (I) are easy to synthesize and have good values of optical energy gap (E_{g}°) and of maximum absorption (λₘₐₓ) in solution, good values of energy levels HOMO (E_{HOMO}) and LUMO (E_{LUMO}) and of electrochemical band-gap (E_{gapEC}), as well as a good solubility in aromatic solvents. Furthermore, said diaryloxybenzoheterodiazole compound disubstituted with thienothiophene groups having general formula (I) can be used as electron acceptor compounds with electron donor compounds having suitable energy levels, i.e. energy levels such as to obtain values of ΔE_{LUMO, D-A} and ΔE_{HOMO, D-A} greater than zero and in any case greater than a threshold value that depends on the pair of electron donor compound (D)—electron acceptor compound (A). In particular, said diaryloxybenzoheterodiazole compound disubstituted with thienothiophene groups having general formula (I) have the following values of the energy levels HOMO (E_{HOMO}) and LUMO (E_{LUMO}): −6.1<E_{HOMO}<−5.5 and −4.1<E_{LUMO}<−3.9 and are, therefore, suitable for use with electron donor compounds having higher energy levels HOMO (E_{HOMO}) and LUMO (E_{LUMO}). Furthermore, said diaryloxybenzoheterodiazole compounds disubstituted with thienothiophene groups having general formula (I) can be advantageously used in organic photovoltaic cells (or solar cells), in particular in binary, ternary, quaternary, organic photovoltaic cells (or solar cells), having both simple and tandem architecture. Furthermore, said diaryloxybenzoheterodiazole compounds disubstituted with thienothiophene groups having general formula (I) can be advantageously used in perovskite-based photovoltaic cells (or solar cells) in the layer based on electron transport material (“Electron Transport Layer—ETL). Moreover, said diaryloxybenzoheterodiazole compounds disubstituted with thienothiophene groups having general formula (I) can be advantageously used in the construction of organic thin film transistors (OTFTs), or of organic field effect transistors (OFETs).

The present disclosure provides a diaryloxybenzoheterodiazole compound disubstituted with thienothiophene groups having general formula (I):

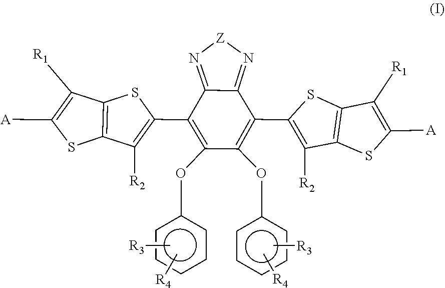

wherein:
Z represents a sulfur atom, an oxygen atom, a selenium atom; or a NR₅ group wherein R₅ is selected from C₁-C₃₀, preferably C₁-C₂₀, linear or branched alkyl groups, or from aryl groups optionally substituted;
R₁ and R₂, identical to or different from each other, represent a hydrogen atom; or are selected from C₁-C₃₀, preferably C₁-C₂₀, linear or branched alkyl groups, optionally containing heteroatoms, cycloalkyl groups optionally substituted, aryl groups optionally substituted, C₁-C₃₀, preferably C₁-C₂₀, linear or branched alkoxyl groups, optionally substituted, —COOR₆ groups wherein R₆ is selected from C₁-C₃₀, preferably C₁-C₂₀, linear or branched alkyl groups, or is a cyano group;
R₃ and R₄, identical to or different from each other, represent a hydrogen atom; or are selected from C₁-C₃₀, preferably C₁-C₂₀, linear or branched alkyl groups, optionally containing heteroatoms, C₁-C₃₀, preferably C₁-C₂₀, linear or branched alkoxyl groups, optionally substituted, —COOR₇ or —OCOR₇ groups wherein R₇ is selected from C₁-C₃₀, preferably C₁-C₂₀, linear or branched alkyl groups, optionally containing heteroatoms, thioether groups —R₈—S—R₉ wherein R₈ and R₉, identical to or different from each other, are selected from C₁-C₃₀, preferably C₁-C₂₀, linear or branched alkyl groups, optionally containing heteroatoms;
A represents an electron acceptor group having general formula (II):

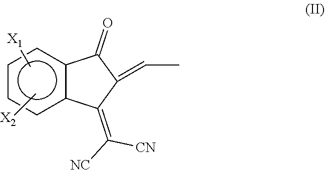

wherein X₁ and X₂, identical to or different from each other, represent a hydrogen atom, or a halogen atom such as, for example, chlorine, fluorine, bromine, preferably chlorine, fluorine; or are selected from C₁-C₃₀, preferably C₁-C₂₀, linear or branched alkyl groups, optionally containing heteroatoms, C₁-C₃₀, preferably C₁-C₂₀, linear or branched alkoxyl groups, optionally substituted; or are selected from —COOR₁₀ ester groups wherein R₁₀ is selected from C₁-C₃₀, preferably C₁-C₂₀, linear or branched alkyl groups, optionally containing heteroatoms, C₁-C₃₀, preferably C₁-C₂₀, linear or branched alkoxyl groups, optionally substituted.

For the purpose of the present description and of the following claims, the definitions of the numerical ranges always include the extremes unless otherwise specified.

For purposes of the present description and of the following claims, the term “comprising” also includes the terms “which essentially consists of” or “which consists of”.

For the purpose of the present description and of the following claims, the term “C₁-C₃₀ alkyl groups” refers to linear or branched alkyl groups having from 1 to 30 carbon atoms. Specific examples of alkyl groups C₁-C₃₀ are: methyl, ethyl, n-propyl, iso-propyl, n-butyl, iso-butyl, t-butyl, pentyl, 2-ethyl-hexyl, hexyl, heptyl, n-octyl, nonyl, decyl, dodecyl, 2-octyldodecyl, 2-butyloctyl, 3,7-dimethyloctyl, 2-octyldecyl, 2-hexyldecyl.

For the purpose of the present description and of the following claims, the term “C₁-C₃₀ alkyl groups optionally containing heteroatoms” means linear or branched alkyl groups having from 1 to 30 carbon atoms, saturated or unsaturated, wherein at least one of the hydrogen atoms is substituted with a heteroatom selected from: halogens such as, for example, fluorine, chlorine, preferably fluorine; nitrogen; sulfur; oxygen. Specific examples of C₁-C₃₀ alkyl groups optionally containing heteroatoms are: fluoromethyl, difluoromethyl, trifluoromethyl, trichloromethyl, 2,2,2-trifluoroethyl, 2,2,2-trichlororoethyl, 2,2,3,3-tetrafluoropropyl, 2,2,3,3,3-pentafluoropropyl, perfluoropentyl, perfluorooctyl, perfluorodecyl, perfluorododecyl, oxymethyl, thiomethyl, thioethyl, dimethylamino, propylamino, dioctylamino.

For the purpose of the present description and of the following claims, the term “cycloalkyl groups” means cycloalkyl groups having from 3 to 20 carbon atoms. Said cycloalkyl groups can optionally be substituted with one or more groups, identical to or different from each other, selected from: halogen atoms such as, for example, fluorine, chlorine, preferably fluorine; hydroxyl groups; C₁-C₃₀ alkyl groups; C₁-C₃₀ alkoxyl groups; cyano groups; amino groups; nitro groups; aryl groups. Specific examples of cycloalkyl groups are: cyclopropyl, 1,4-dioxino, 2,2-difluorocyclopropyl, cyclobutyl, cyclopentyl, cyclohexyl, methylcyclohexyl, methoxycyclohexyl, fluorocyclohexyl, phenylcyclohexyl.

For the purpose of the present description and of the following claims, the term “aryl groups” refers to aromatic carbocyclic groups having from 6 to 60 carbon atoms. Said aryl groups can optionally be substituted with one or more groups, identical to or different from each other, selected from: halogen atoms such as, for example, fluorine, chlorine, preferably fluorine; hydroxyl groups; C₁-C₃₀ alkyl groups; C₁-C₃₀ alkoxyl groups; cyano groups; amino groups; nitro groups; aryl groups, phenoxyl groups. Specific examples of aryl groups are: phenyl, methylphenyl, 2,5-dimethylphenyl, 2,6-dimethylphenyl, 2,4,6-triphenoxyphenyl, trimethylphenyl, di-iso-propylphenyl, t-butylphenyl, methoxyphenyl, hydroxyphenyl, 2-phenoxyphenyl, fluorophenyl, pentafluorophenyl, chlorophenyl, nitrophenyl, dimethylaminophenyl, naphthyl, phenylnaphthyl, phenanthrene, anthracene.

For the purpose of the present description and of the following claims, the term “heteroaryl groups” means aromatic, penta- or hexa-atomic heterocyclic groups, also benzocondensed or heterobicyclic, containing from 4 to 60 carbon atoms and from 1 to 4 heteroatoms selected from nitrogen, oxygen, sulfur, silicon, selenium, phosphorus. Said heteroaryl groups can optionally be substituted with one or more groups, identical to or different from each other, selected from: halogen atoms such as, for example, fluorine, chlorine, bromine, preferably fluorine; hydroxyl groups; C₁-C₁₂ alkyl groups; C₁-C₁₂ alkoxyl groups; C₁-C₁₂ thioalkoxyl groups; C₃-C₂₄ tri-alkylsilyl groups; polyethyleneoxyl groups; cyano groups; amino groups; C₁-C₁₂ mono- or di-alkylamine groups; nitro groups. Specific examples of heteroaryl groups are: pyridine, methylpyridine, methoxypyridine, phenylpyridine, fluoropyridine, pyrimidine, pyridazine, pyrazine, triazine, tetrazine, quinoline, quinoxaline, quinazoline, furan, thiophene, hexylthiophene, bromothiophene, dibromothiophene, pyrrole, oxazole, thiazole, isoxazole, isothiazole, oxadiazole, thiadiazole, pyrazole, imidazole, triazole, tetrazole, indole, benzofuran, benzothiophene, benzooxazole, benzothiazole, benzooxadiazole, benzothiadiazole, benzopyrazole, benzimidazole, benzotriazole, triazolepyridine, triazolepyrimidine, cumarine.

For the purpose of the present description and of the following claims, the term “C₁-C₃₀ alkoxyl groups” means linear or branched alkoxyl groups having from 1 to 30 carbon atoms. Said alkoxyl groups can optionally be substituted with one or more groups, identical to or different from each other, selected from: halogen atoms such as, for example, fluorine, chlorine, preferably fluorine; hydroxyl groups; C₁-C₃₀ alkyl groups; C₁-C₃₀ alkoxyl groups; cyano groups; amino groups; nitro groups. Specific examples of C₁-C₂₀ alkoxyl groups are: methoxyl, ethoxyl, fluoroethoxyl, n-propoxyl, iso-propoxyl, n-butoxyl, n-fluoro-butoxyl, iso-butoxyl, t-butoxyl, pentoxyl, hexyloxyl, heptyloxyl, octyloxyl, nonyloxyl, decyloxyl, dodecyloxyl.

According to a preferred embodiment of the present disclosure, in said general formula (I):
Z represents a sulfur atom;
R₁ and R₂, identical to each other, represent a hydrogen atom; or R₁ and R₂, different from each other, represent a hydrogen atom or a C₁-C₃₀ alkyl group, preferably R₁ represents n-octyl and R₂ represents a hydrogen atom;
R₃ and R₄, identical to or different from each other, represent a hydrogen atom, or represent a —COOR₇ group wherein R₇ represents a C₁-C₃₀ alkyl group, preferably 2-octyldodecyl, 2-hexyldecyl, 2-butyloctyl, said —COOR₇ group being in position 2, or in position 3, or in position 4, or in position 3,5 of the phenyl;
A represents an electron acceptor group having general formula (II) wherein X₁ and X₂, identical to each other, represent a hydrogen atom, a fluorine atom, or a chlorine atom.

Specific examples of diaryloxybenzoheterodiazole compound disubstituted with thienothiophene groups having general formula (I) useful for the purpose of the present disclosure are reported in Table 1.

TABLE 1

| | |
|---|---|
| 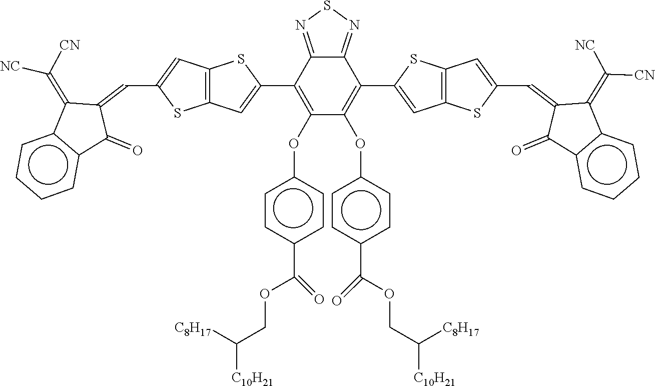 | GS7 |
| 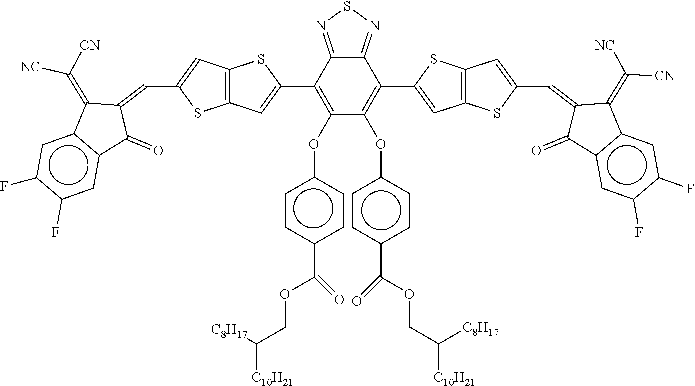 | GS8 |
| 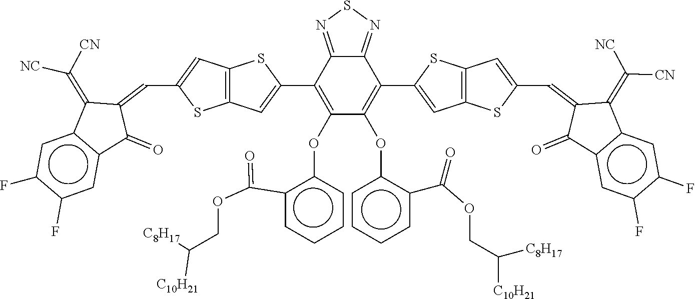 | GS12 |
| 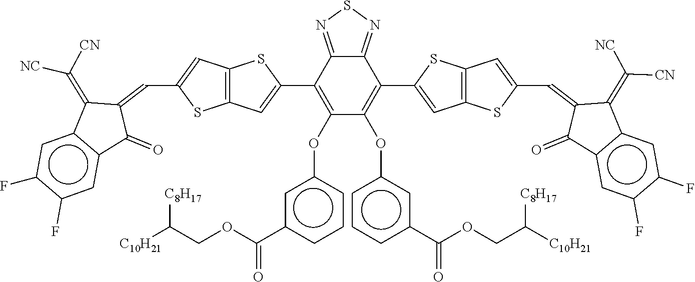 | GS13 |
| 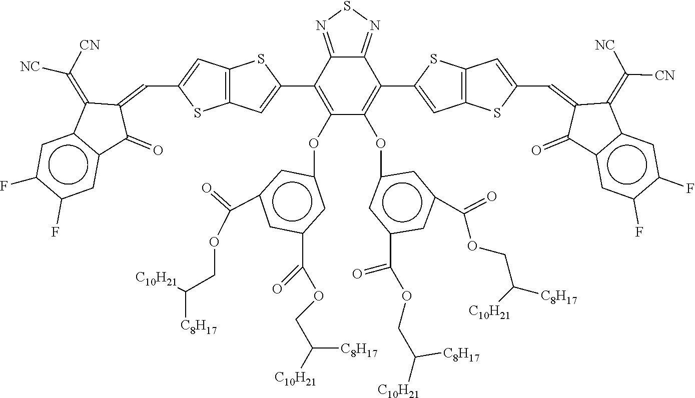 | GS14 |
| 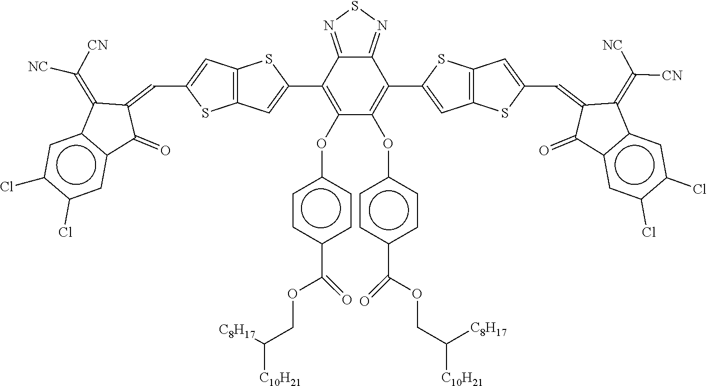 | GS27 |

The diaryloxybenzoheterodiazole compound disubstituted with thienothiophene groups having general formula (I) of the present disclosure can be obtained by processes known in the art as reported, for example, in the international patent application WO 2016/046319 in the name of the Applicant and incorporated herein as reference, or through the processes reported below.

For example, 4,7-dibromo-5,6-difluorobenzothiadiazole (4) is reacted with 2-octyldodecyl4-hydroxybenzoate (3), in a basic environment, to give the nucleophilic substitution reaction, wherein the atoms of fluorine are easily substituted with a nucleophile: in this case, the phenate ion is prepared in situ by the reaction of a phenol with a base such as, for example, potassium carbonate (K₂CO₃) (Scheme 1). The reaction can be carried out in dipolar aprotic solvents, for example, N,N-dimethylformamide (DMF), or in the presence of crown ethers, as reported, for example in the international patent application WO 2019/138332 in the name of the Applicant and incorporated herein by reference.

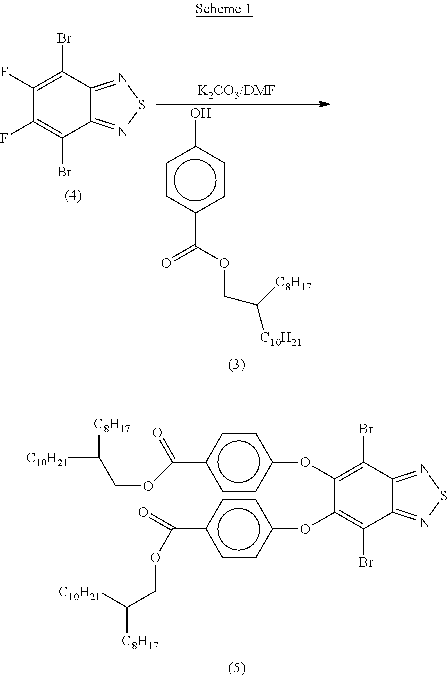

The 2-octyldodecyl 4-hydroxybenzoate (3), is not a commercially available product and can be obtained starting from potassium 4-hydroxybenzoate, obtained in situ by reacting 4-hydroxybenzoic acid (1) with potassium bicarbonate (KHCO₃), by alkylation with 2-octyldodecylbromide (2), in the presence of potassium iodide: the reaction is carried out at 80° C., in a dipolar aprotic solvent, for example N,N-dimethylformamide (DMF), as reported in Scheme 2.

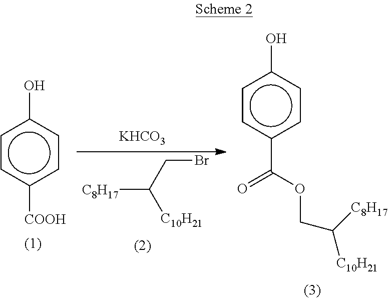

In particular, in order to obtain the diaryloxybenzoheterodiazole compounds disubstituted with thienothiophene groups (GS7) and (GS8), 4,7-dibromo-5,6-di (4-carbo(2-octyldodecyloxy)phenoxy)benzothiadiazole (5) is reacted with 2-tributylstannylthienothiophene (7), by Stille reaction, in the presence of palladium catalysts Pd (II) or Pd (0) such as, for example, Pd(PPh₃)₄[Ding L. et al., “Angewandte Chemie Int. Ed.”, (2012), Vol. 51, p. 9038-9041], Pd(OAc)₂ [Xie Y—X. et al., “Tetrahedron” (2006), Vol. 62, p. 31-38], Pd(PPh₃)₂C₁₂ (Caccialli F. et al., “Chemical Communications” (2011), Vol. 47, Pag. 8820-8822), Pd₂dba₃/P(o-tol)₃ [Reynolds J. R. et al., “Journal of American Chemical Society” (2012), Vol. 134, p. 2599-2612]. For the purpose of the present disclosure, the pair consisting of Pd₂dba₃/P(o-tol)₃ was used as catalyst obtaining 4,7-di(2-thienothienyl)-benzothiadiazole (8) (Scheme 3): the reaction was carried out in toluene, at 108° C., and the compound obtained at the end of the reaction was isolated by elution on a silica gel chromatographic column. The 2-tributylstannylthienothiophene (7) used as a reagent, must be previously prepared by means of the metalation reaction of thienothiophene (6), a commercially available product: this reaction was carried out in tetrahydrofuran, at −78° C., in the presence of a stoichiometric quantity of a base that can be selected, for example, between n-butylithium (Kawabata K. et al., “Macromolecules” (2013), Vol. 46, p. 2078-2091), lithium diisopropylamide (LDA) (Wu Y. et al., “Journal of Material Chemistry” (2012), Vol. 22, p. 21362-21365). For the purpose of the present disclosure, n-butylithium (BuLi) (solution 1.6 M in hexane) was used. The lithium salt thus obtained was reacted directly with tributylstannylchloride to give 2-tributylstannylthienothiophene (7): at the end of the reaction, the obtained mixture was washed with a saturated aqueous solution of sodium bicarbonate. After removing the solvent, by distillation at reduced pressure, 2-tributistannylthienothiophene (7) was used, without any purification, in the Stille reaction (Scheme 3).

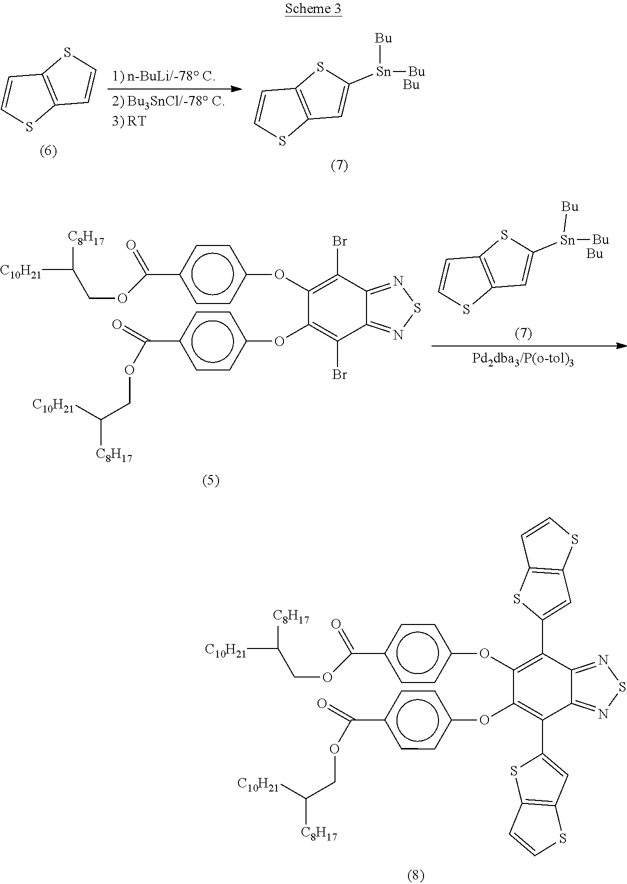

wherein RT=room temperature.

The 4,7-di(2-thienothienyl)-5,6-di[4-carbo(2-octyldodecyloxy)phenoxy]-benzothiadiazole (8) obtained as described above, was subjected to formylation by the Vilsmeier-Haak reaction, [Lee J. K. et al., “Journal of Photochemistry and Photobiology A: Chemistry” (2014), Vol. 275, p. 47-53], obtaining 4,7-di(2-(5-formyl-thienothienyl)-5,6-di[4-carbo(2-octyldodecyloxy)phenoxy]benzo-thiadiazole (9). The formylating agent was prepared in situ by the reaction of N,N-dimethylformamide (DMF) and phosphorus oxychloride (POCl₃) to give the iminium ion: at the end of the reaction, the mixture obtained was subjected to aqueous quenching in the presence of potassium acetate to hydrolyze the species formed by the electrophilic attack and thus give the corresponding aldehyde, as reported in Scheme 4.

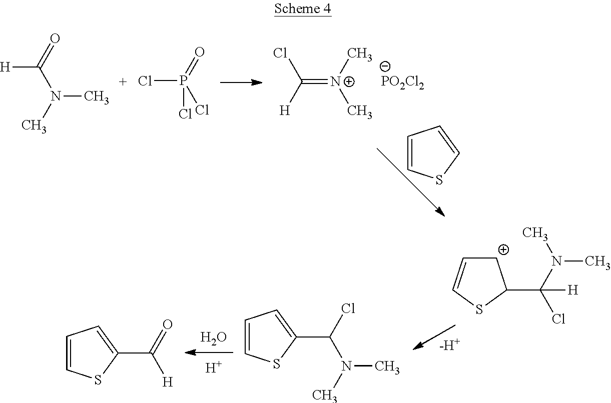

The formylating agent, obtained in situ according to the reaction reported in Scheme 4, was reacted with 4,7-di(2-thienothienyl)-5,6-di[4-carbo(2-octyldodecyloxy)phenoxy]benzothiadiazole (8), in the presence of chloroform (CHCl₃) (Lee J. et al., “Journal of Photochemistry And Photobiology. A: Chemistry” (2014), Vol. 275, pages 47-53), obtaining the 4,7-di(2-(5-formyl-2-thienothienyl)-5,6-di[4-carbo(2-octyldodecyloxy)phenoxy]benzothiadiazole (9) after quenching with an aqueous solution of potassium acetate and purification by elution on a silica gel chromatographic column (Scheme 5).

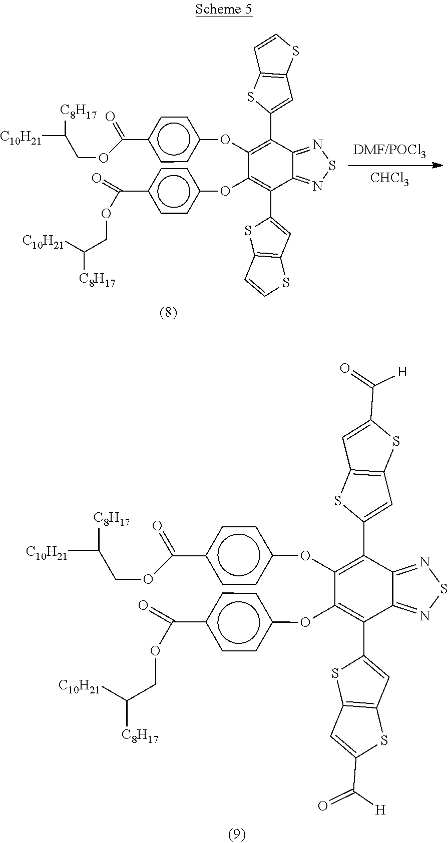

The 4,7-di(2-(5-formyl-2-thienothienyl)-5,6-di[4-carbo(2-octyldodecyl-oxy)-phenoxy]-benzothiadiazole (9) obtained as reported in Scheme 5, was reacted with active methylene compounds, by Knovenagel reaction [Gao C. et al., “Dyes and Pigments” (2020), Vol. 178, 108388]: in particular, with 3-(dicyanomethylidene) indan-1-one (10) and with 5,6-difluoro-3-(dicyanomethylidene)indan-1-one (11), which by reacting, in the presence of chloroform (CHCl₃), in a basic environment in the presence of an excess of pyridine (Py), and eliminating of the air present in the reaction environment by means of vacuum/argon cycles, give respectively compounds GS7, GS8 and GS27 (Scheme 6).

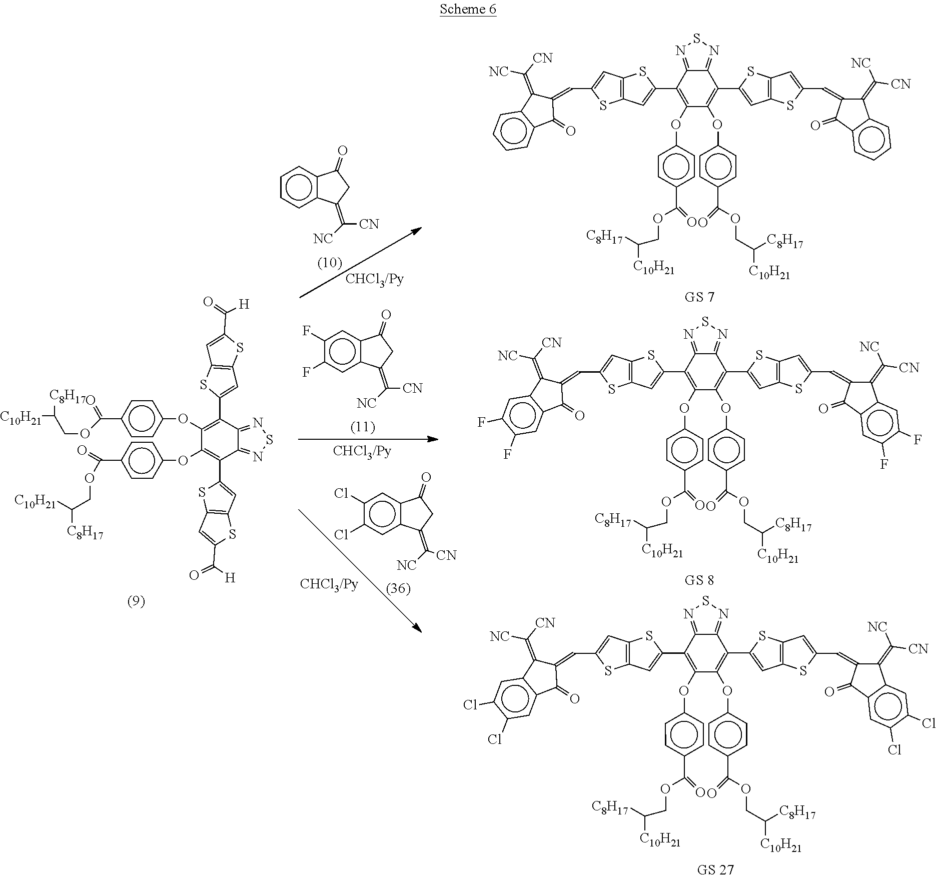

The compounds GS12, GS13, GS14, have been synthesized in a similar way of the compound GS8, replacing only the 2-octyldodecyl 4-hydroxybenzoate (3) with: a) 2-octyldodecyl 2-hydroxybenzoate (18), obtained from alkylation of 2-hydroxybenzoic acid (17), to obtain compound GS12, b) 2-octyldodecyl 3-hydroxybenzoate (20), obtained from the alkylation of 3-hydroxybenzoic acid (19), to obtain compound GS13, c) 2-octyldodecyl 3-hydroxyisophthalate (22), obtained from the alkylation of isophthalic acid (21), to obtain compound GS14.

Further details relating to the processes for the preparation of said diaryloxybenzoheterodiazole compound disubstituted with thienothiophene groups having general formula (I) can be found in the following examples.

As described above, said diaryloxybenzoheterodiazole compound disubstituted with thienothiophene groups having general formula (I) can be advantageously used as an electron acceptor compound in organic photovoltaic devices (or solar devices) selected, for example, from binary, ternary, quaternary, organic photovoltaic cells (or solar cells), having both simple and tandem architecture, organic photovoltaic modules (or solar modules), both on rigid support and on flexible support.

Consequently, the present disclosure provides an organic photovoltaic device (or solar device) selected, for example, from binary, ternary quaternary, organic photovoltaic cells (or solar cells) having both simple and tandem architecture, organic photovoltaic modules (or solar modules), both on a rigid support and on a flexible support, comprising at least one diaryloxybenzoheterodiazole compound disubstituted with thienothiophene groups having general formula (I), preferably a binary, ternary, quaternary, organic photovoltaic cell (or solar cell), having both simple and tandem architecture.

According to a preferred embodiment of the present disclosure, said binary, ternary, quaternary, organic photovoltaic cell (or solar cell), having both simple and tandem architecture, comprises:
at least one rigid or flexible support;
an anode;
at least one layer of photoactive material;
a cathode;
   wherein said layer of photoactive material comprises at least one photoactive organic polymer as electron donor compound, at least one diaryloxybenzoheterodiazole compound disubstituted with thienothiophene groups having general formula (I) as electron acceptor compound.

According to a preferred embodiment of the present disclosure, said photoactive organic polymer can be selected, for example, from:
(a) polythiophenes such as, for example, regioregular poly(3-hexylthiophene) (P3HT), poly(3-octylthiophene), poly(3,4-ethylenedioxythiophene), or mixtures thereof;
(b) alternating or statistical conjugated copolymers comprising:
   at least one benzotriazole unit (B) having general formula (Ia) or (Ib):

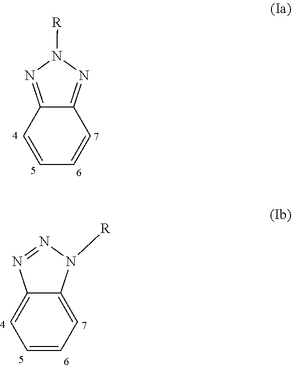

wherein R group is selected from alkyl groups, aryl groups, acyl groups, thioacyl groups, said alkyl, aryl, acyl and thioacyl groups being optionally substituted;
at least one conjugated structural unit (A), wherein each unit (B) is connected to at least one unit (A) in any one of the positions 4, 5, 6, or 7, preferably in the positions 4 or 7;
(c) alternating conjugated copolymers comprising benzothiadiazole units such as, for example, PCDTBT {poly[N-9″-heptadecanyl-2,7-carbazole-alt-5,5-(4′,7′-di-2-thienyl-2′,1′,3′-benzothiadiazole]}, PCPDTBT {poly[2,6-(4,4-bis-(2-ethylhexyl)-4H-cyclopenta[2,1-b;3,4-b′]-dithiophene)-alt-4,7-(2,1,3-benzothiadiazole)]};
(d) alternating conjugated copolymers comprising thieno[3,4-b]pyrazidine units;
(e) alternating conjugated copolymers comprising quinoxaline units; (f) alternating conjugated copolymers comprising silolics monomer units such as, for example, 9,9-dialkyl-9-silafluorene copolymers;
(g) alternating conjugated copolymers comprising condensed thiophene units such as, for example, copolymers of thieno[3,2-b]thiophene and of benzo[1,2-b:4,5-b′]dithiophene;
(h) alternating conjugated copolymers comprising benzothiadiazole or naphthothiadiazole units substituted with at least one fluorine atom and thiophene units substituted with at least one fluorine atom such as, for example, PffBT4T-2OD {poly[(5,6-difluoro-2,1,3-benzothiadiazole-4,7-diyl)-alt-(3,3′″-di(2-octyldodecyl)-2,2′,5′,2″,5″,2′″-quaterthiophene-5,5′″-diyl)]}, PBTff4T-2OD {poly[(2,1,3-benzothiadiazole-4,7-diyl)-alt-(4′,3″-difluoro-3,3′″-di(2-octyldodecyl)-2,2′;5′,2″;5″,2′″-quaterthiophene-5,5′″-diyl)]}, PNT4T-2OD {poly(naphtho[1,2-c:5,-c′]bis[1,2,5]thiadiazole-5,10-diyl)-alt-(3,3′″-di(2-octyldodecyl)-2,2′;5′,2″;5″,2′″-quaterthiophene-5,5′″-diyl)]};
(i) conjugated copolymers comprising thieno[3,4-c]pyrrole-4,6-dione units such as, for example, PBDTTPD {poly[[5-(2-ethylhexyl)-5,6-dihydro-4,6-dioxo-4H-thieno[3,4-c]pyrrole-1,3-diyl][4,8-bis[(2-ethylhexyl)oxy]benzo[1,2-b:4,5-b′]dithiophene-2,6-diyl]};
(l) conjugated copolymers comprising thienothiophenic units such as, for example, PTB7 {poly({4,8-bis[(2-ethylhexyl)oxy]benzo[1,2-b:4,5-b′]dithiophene-2,6-diyl}{3-fluoro-2-[(2-ethylhexyl)carbonyl]thieno[3,4-b]thiophenediyl}}, PBDB-T polymer poly[[4,8-bis[5-(2-ethylhexyl)-2-thienyl]benzo[1,2-b:4,5-b′]dithiophene-2,6-diyl]-2,5-thiophenediyl[5,7-bis(2-ethylhexyl)-4,8-dioxo-4H,8H-benzo[1,2-c:4,5-c′]dithiophene-1,3-diyl]-2,5-thiophenediyl];
(m) polymers comprising a derivative of indacen-4-one having general formula (III), (IV) or (V):

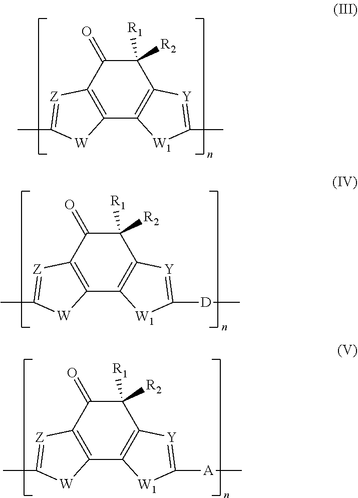

wherein:
   W and W₁, identical to or different from each other, preferably identical to each other, represent an oxygen atom; a sulfur atom; an N—R₃ group wherein R₃ represents a hydrogen atom, or is selected from C₁-C₂₀, preferably C₂-C₁₀, linear or branched alkyl groups;
   Z and Y, identical to or different from each other, preferably identical to each other, represent a nitrogen atom; or a CR₄ group wherein R₄ represents a hydrogen atom, or is selected from C₁-C₂₀, preferably C₂-C₁₀, linear or branched alkyl groups, cycloalkyl groups optionally substituted, aryl groups optionally substituted, heteroaryl groups optionally substituted, C₁-C₂₀, preferably C₂-C₁₀, linear or branched alkoxyl groups, polyethyleneoxyl groups R₅—O—[CH₂—CH₂—O]ₙ— wherein R₅ is selected from C₁-C₂₀, preferably C₂-C₁₀, linear or branched alkyl groups, and n is an integer ranging from 1 to 4, —R₆—OR₇ groups wherein R₆ is selected from C₁-C₂₀, preferably C₂-C₁₀, linear or branched alkylene groups and R₇ represents a hydrogen atom or is selected from C₁-C₂₀, preferably C₂-C₁₀, linear or branched alkyl groups, or is selected from polyethyleneoxyl groups R₅—[—OCH₂—CH₂—O]ₙ— wherein R₅ has the same meanings reported above and n is an integer ranging from 1 to 4, —COR₈ groups wherein R₈ is selected from C₁-C₂₀, preferably C₂-C₁₀, linear or branched alkyl groups, —COOR₉ groups wherein R₉ is selected from C₁-C₂₀, preferably C₂-C₁₀, linear or branched alkyl groups; or represent a —CHO group, or a cyano group (—CN);
   R₁ and R₂, identical to or different from each other, preferably identical to each other, are selected from C₁-C₂₀, preferably C₂-C₁₀, linear or branched alkyl groups; cycloalkyl groups optionally substituted; aryl groups optionally substituted; heteroaryl groups optionally substituted; C₁-C₂₀, preferably C₂-C₁₀, linear or branched alkoxyl groups; polyethyleneoxyl groups R₅—O—[CH₂—CH₂—O]ₙ— wherein R₅ has the same meanings reported above and n is an integer ranging from 1 to 4; —R₆—OR₇ groups wherein R₆ and R₇ have the same meanings reported above; —COR₈ groups wherein R₈ has the same meanings reported above; —COOR₉ groups wherein R₉ has the same meanings reported above; or represent a —CHO group, or a cyano group (—CN);
   D represents an electron-donor group;
   A represents an electron acceptor group;
   n is an integer ranging from 10 to 500, preferably ranging from 20 to 300;
(n) polymers comprising antradithiophene derivatives having general formula (X):

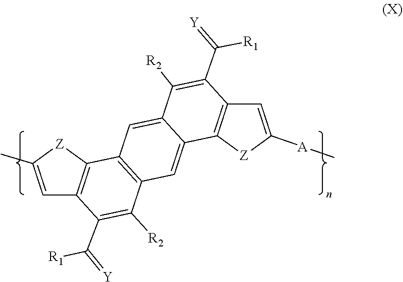

wherein:
   Z, identical to or different from each other, preferably identical to each other, represent a sulfur atom, an oxygen atom, a selenium atom;
   Y, identical to or different from each other, preferably identical to each other, represent a sulfur atom, an oxygen atom, a selenium atom;
   R₁, identical to or different from each other, preferably identical to each other, are selected from amino groups —N—R₃R₄ wherein R₃ represents a hydrogen atom, or is selected from C₁-C₂₀, preferably C₂-C₁₀, linear or branched alkyl groups, or is selected from cycloalkyl groups optionally substituted and R₄ is selected from C₁-C₂₀, preferably C₂-C₁₀, linear or branched alkyl groups, or is selected from cycloalkyl groups optionally substituted; or are selected from C₁-C₃₀, preferably C₂-C₂₀, linear or branched alkoxy groups; or are selected from polyethyleneoxyl groups R₅—O—[CH₂—CH₂—O]ₙ— wherein R₅ is selected from C₁-C₂₀, preferably C₂-C₁₀, linear or branched alkyl groups, and n is an integer ranging from 1 to 4; or are selected from —R₆—OR₇ groups wherein R₆ is selected from C₁-C₂₀, preferably C₂-C₁₀, linear or branched alkylene groups and R₇ represents a hydrogen atom, or is selected from C₁-C₂₀, preferably C₂-C₁₀, linear or branched alkyl groups, or is selected from polyethyleneoxyl groups R₅—[—OCH₂—CH₂-]ₙ—wherein R₅ has the same meanings reported above and n is an integer ranging from 1 to 4; or are selected from thiol groups —S—R₅ wherein R₅ is selected from C₁-C₂₀, preferably C₂-C₁₀, linear or branched alkyl groups;
   R₂, identical to or different from each other, preferably identical to each other, represent a hydrogen atom; or are selected from C₁-C₂₀, preferably C₂-C₁₀, linear or branched alkyl groups; or are selected from —COR₉ groups wherein R₉ is selected from C₁-C₂₀, preferably C₂-C₁₀, linear or branched alkyl groups; or are selected from —COOR₁₀ groups wherein R₁₀ is selected from C₁-C₂₀, preferably C₂-C₁₀, linear or branched alkyl groups; or are selected from aryl groups optionally substituted; or are selected from heteroaryl groups optionally substituted;
A represents an electron-acceptor group;
n is an integer ranging from 10 to 500, preferably from 20 to 300.

More details relating to the alternating or statistical conjugated copolymers (b) comprising at least one benzotriazole unit (B) and at least one conjugated structural unit (A) and to the process for their preparation can be found, for example, in the international patent application WO 2010/046114 in the name of the Applicant.

More details relating to alternating conjugated copolymers comprising benzothiadiazole units (c), alternating conjugated copolymers comprising thieno[3,4-b]pyrazidine units (d), alternating conjugated copolymers comprising quinoxaline units (e), alternating conjugated copolymers comprising silolic monomer units (f), alternating conjugated copolymers comprising condensed thiophenic units (g), can be found, for example, in Chen J. et al., “Accounts of Chemical Research” (2009), Vol. 42, No. 11, p. 1709-1718; Pò R. et al., “Macromolecules” (2015), Vol. 48 (3), p. 453-461.

More details relating to alternating conjugated copolymers comprising benzothiadiazole or naphthothiadiazole units substituted with at least one fluorine atom and thiophene units substituted with at least one fluorine atom (h) can be found, for example, in Liu Y. et al., “Nature Communications” (2014), Vol. 5, Article no. 5293 (DOI:10.1038/ncomms6293).

More details regarding conjugated copolymers comprising thieno[3,4-c]pyrrole-4,6-dione (i) units can be found, for example, in Pan H. et al, “Chinese Chemical Letters” (2016), Vol. 27, Issue 8, p. 1277-1282.

More details regarding conjugated copolymers comprising thienotiophenic units (l) can be found, for example in Liang Y. et al., “Journal of the American Chemical Society” (2009), Vol. 131 (22), p. 7792-7799; Liang Y. et al., “Accounts of Chemical Research” (2010), Vol. 43 (9), p. 1227-1236.

More details relating to the polymers comprising a derivative of indacen-4-one (m) can be found, for example, in the international patent application WO 2016/180988 in the name of the Applicant.

More details relating to polymers comprising antradithiophenic derivatives having general formula (X) (n) can be found, for example, in the international patent application WO 2019/175367 in the name of the Applicant.

According to a preferred embodiment of the present disclosure, in the case of a ternary organic photovoltaic cell (or solar cell), having both simple and tandem architecture, the photoactive layer can comprise, for example:
two photoactive organic polymers selected from those reported above and one diaryloxybenzoheterodiazole compound disubstituted with thienothiophene groups having general formula (I); or
one photoactive organic polymer selected from those reported above and two diaryloxybenzoheterodiazole compound disubstituted with thienothiophene groups having general formula (I); or
one photoactive organic polymer selected from those reported above, one diaryloxybenzoheterodiazole compound disubstituted with thienothiophene groups having general formula (I) and one fullerene derivative such as, for example PC61BM (6,6-phenyl-C₆₁-methyl butyric ester) or the PC71BM (6,6-phenyl-C₇₁-methyl butyric ester); or
one photoactive organic polymer selected from those reported above, one diaryloxybenzoheterodiazole compound disubstituted with thienothiophene groups having general formula (I) and one non-fullerene compound selected, for example, from non-fullerene compounds, possibly polymeric, such as, for example, compounds based on perylene-diimides or naphthalene-diimides and fused aromatic rings; indacenothiophenes with electron-poor terminal groups; compounds having an aromatic core capable of symmetrically rotating, for example, derivatives of corannulene or truxenone.

According to a preferred embodiment of the present disclosure, in the case of a quaternary organic photovoltaic cell (or solar cell), having both simple and tandem architecture, the photoactive layer can comprise, for example:
two photoactive organic polymers selected from those reported above and two diaryloxybenzoheterodiazole compound disubstituted with thienothiophene groups having general formula (I); or
two photoactive organic polymers selected from those reported above, one diaryloxybenzoheterodiazole compound disubstituted with thienothiophene groups having general formula (I) and two fullerene derivative such as, for example PC61BM (6,6-phenyl-C₆₁-methyl butyric ester) or PC71BM (6,6-phenyl-C₇₁-methyl butyric ester); or
two photoactive organic polymers selected from those reported above, one diaryloxybenzoheterodiazole compound disubstituted with thienothiophene groups having general formula (I) and one non-fullerene compound selected, for example, from non-fullerene compounds, possibly polymeric, such as, for example, compounds based on perylene-diimides or naphthalene-diimides and fused aromatic rings; indacenothiophenes with electron-poor terminal groups; compounds having an aromatic core capable of symmetrically rotating, for example, derivatives of corannulene or truxenone.

Specific examples of said non-fullerene, optionally polymeric compounds are: 3,9-bis(2-methylene-[3-(1,1-dicyanomethylene)-6,7-difluoro)-indanone))-5,5,11,11-tetrakis(4-hexylphenyl)-dithieno[2,3-d:2′,3‘-d’]-s-indaceno-[1,2-b:5,6-b′]-dithiophene, poly{[N,N-bis(2-octyldodecyl)-1,4,5,8-naphthalene-diimide-2,6-diyl]-alt-5,5′-(2,2′-bithiophene)}, 2,2′-((2Z,2′Z)-((4,4,9,9-tetrahexyl-4,9-dihydro-s-indacene[1,2-b:5,6-b′]dithiophene-2,7-diyl)bis(methanylidene))bis-(3-oxo-2,3-dihydro-1H-indene-2,1-diylidene))dimalononitrile, 2,2′-[[6,6,12,12-tetrakis(4-hexylphenyl)-6,12-dihydroditheno[2,3-d:2′,3′-d]-s-indacene[1,2-b:5,6-b′]dithiophene-2,8-diyl]bis[methylidene(3-oxo-1H-indene-2,1(3H)-diylidene)]]-bis[propanodinitrile](ITIC), ITIC-4F.

More details relating to said non-fullerene compounds can be found, for example, in Nielsen C. B. et al, “Accounts of Chemical Research” (2015), Vol. 48, p. 2803-2812; Zhan C. et al, “RSC Advances” (2015), Vol. 5, p. 93002-93026; Lin Y. et al. “Advanced Materials” (2015), Vol. 27, Issue 7, p. 1170-1174, above.

The aforementioned organic photovoltaic cell (or solar cell) can be obtained according to the methods known in the art reported above.

### DESCRIPTION OF THE DRAWINGS AND DISCLOSURE

The present disclosure will now be illustrated in greater detail through an embodiment with reference to FIG. 1 below.

FIG. 1 represents a cross-sectional view of an organic photovoltaic cell (or solar cell) of the present disclosure.

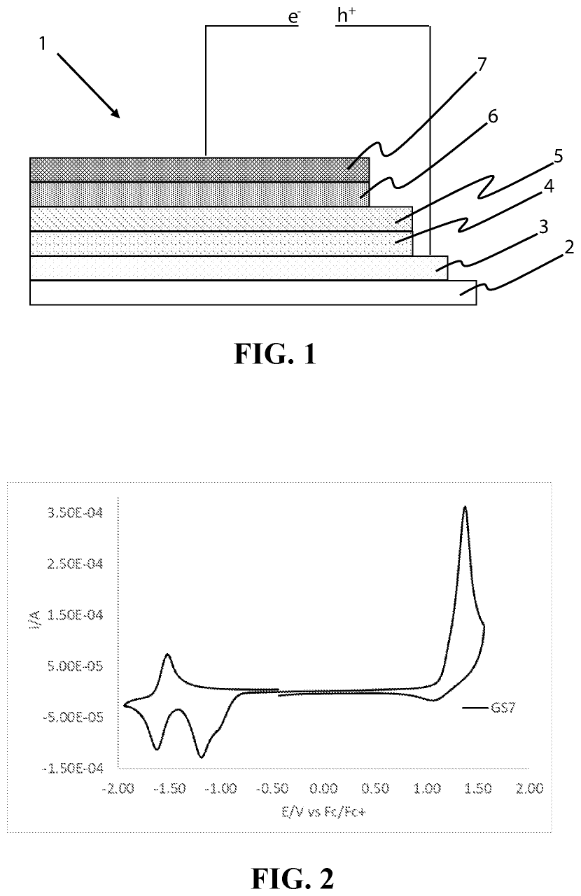

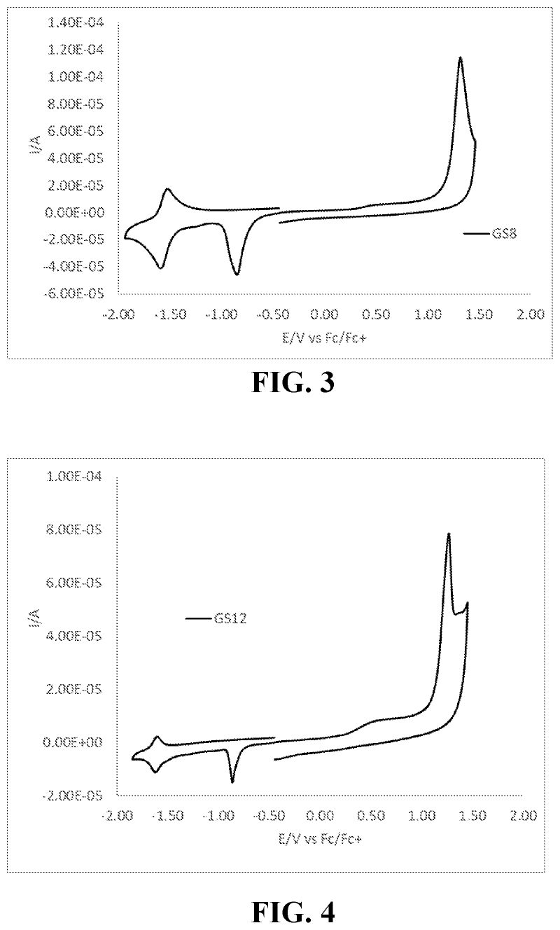

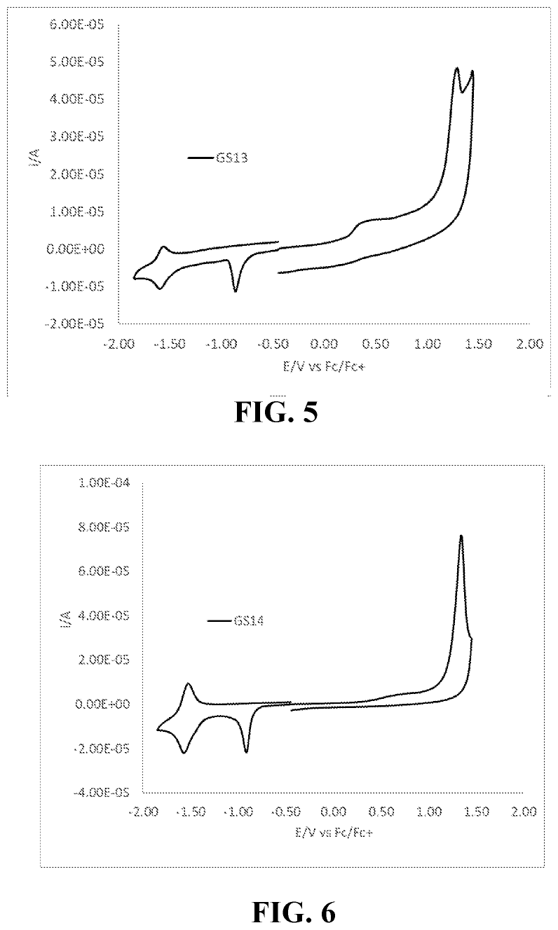

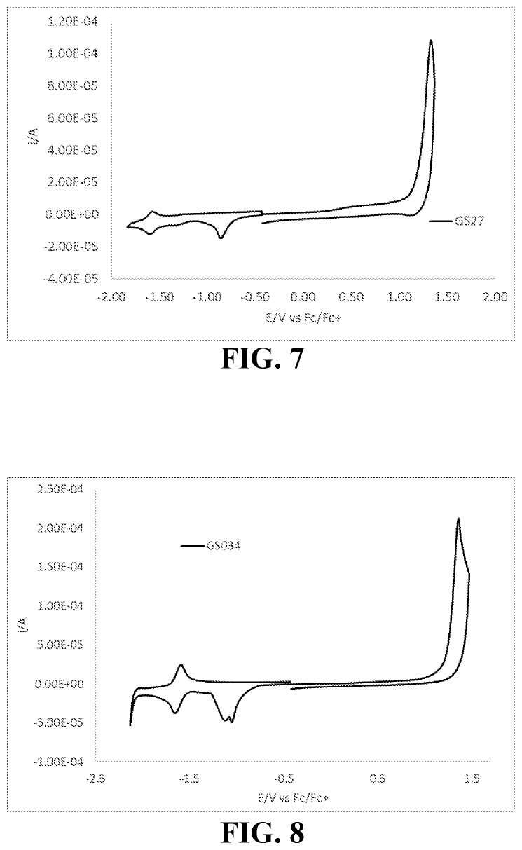

With reference to FIG. 1, the organic photovoltaic cell (or solar cell) (1) having a bulk heterojunction architecture comprises:
a transparent support (2) of glass or plastic such as, for example, polyethylene terephthalate (PET), polyethylene naphthalate (PEN), polycarbonate (PC), polypropylene (PP), polyimide (PI), triacetyl cellulose (TAC), or their copolymers;
an anode (3), preferably an indium tin oxide (ITO)] anode;
a layer of PEDOT:PSS[poly(3,4-ethylenedioxythiophene)polystyrene sulfonate](4);
a layer of photoactive material (5) comprising at least one photoactive organic polymer, at least one diaryloxybenzoheterodiazole compound disubstituted with thienothiophene groups having general formula (I);
a cathode buffer layer (6), preferably comprising lithium fluoride;
a cathode (7), preferably an aluminum cathode.

As described above, said diaryloxybenzoheterodiazole compound disubstituted with thienothiophene groups having general formula (I) can be advantageously used in perovskite-based photovoltaic cells (or solar cells) in the layer based on electron transport material (Electron Transport Layer—ETL).

Consequently, the present disclosure provides a perovskite-based photovoltaic cell (or solar cell) wherein the layer based on electron transport material (Electron Transport Layer—ETL) comprises at least one diaryloxybenzoheterodiazole compound disubstituted with thienothiophene groups having general formula (I).

As described above, said diaryloxybenzoheterodiazole compound disubstituted with thienothiophene groups having general formula (I) can be advantageously used in the construction of organic thin film transistors (OTFTs), or of organic field effect transistors (OFETs).

Consequently, the present disclosure provides organic thin film transistors (OTFTs), or organic field effect transistors (OFETs), comprising at least one diaryloxybenzoheterodiazole compound disubstituted with thienothiophene groups having general formula (I).

In the following examples, the analytical techniques and characterization methodologies reported below were used.

### ¹H-NMR Spectra

The ¹H-NMR spectra were recorded by means of a nuclear magnetic resonance spectrometer mod. Bruker Avance 400, using deuterated chloroform (CDCl₃) at 25° C. and tetramethylsilane (TMS) (Merck) as internal standard. For this purpose, solutions of the diaryloxybenzoheterodiazole compounds disubstituted with thienothiophene groups having general formula (I) of the present disclosure, obtained in accordance with the following examples, having concentrations equal to 10% by weight with respect to the total weight of the final solution, were used.

### Absorption Spectra

The absorption spectra of the o-xylene or dichlorobenzene solutions of the diaryloxybenzoheterodiazole compound disubstituted with thienothiophene groups having general formula (I) of the present disclosure obtained in accordance with the following examples, in the ultraviolet and visible (UV-Vis) (250 nm-800 nm), were acquired in transmission using a Perkin Elmer λ 950 double beam and double monochromator spectrophotometer, with a 2.0 nm bandwidth and 1.0 nm step, using quartz cuvettes with optical path equal to 1 cm. The respective optical energy gaps (E_{g}°) were also determined from these spectra, using the tangent method.

Determination of HOMO (E_{HOMO}) and LUMO (E_{LUMO}) Energy Levels

The determination of the values of HOMO (E_{HOMO}) and LUMO (E_{LUMO}) energy levels of the diaryloxybenzoheterodiazole compound disubstituted with thienothiophene groups having general formula (I) of the present disclosure obtained by operating in accordance with the following examples, was carried out by means of the cyclic voltammetry (CV) technique. With this technique it is possible to measure the values of the radical cation formation potentials and the radical anion formation potentials of the sample in question. From the values of the potentials thus obtained, it is possible to extrapolate the values of the HOMO (E_{HOMO}) and LUMO (E_{LUMO}) energy levels of the sample under examination (expressed in eV). The difference between the values of the HOMO (E_{HOMO}) and LUMO (E_{LUMO}) energy levels gives the value of the electrochemical band-gap (E_{gapEC}).

The values of the electrochemical band-gap (E_{gapEC}) are generally higher than the values of the optical energy-gap (E_{g}°) since during the execution of the cyclic voltammetry (CV), the neutral compound is charged and it undergoes a conformational reorganization, with an increase in the energy gap, while the optical measurement does not lead to the formation of charged species.

The cyclic voltammetry (CV) measurements were performed with an Autolab PGSTAT12 potentiostat (with GPES Ecochemie software) in a three-electrode cell. In the measurements carried out, an Ag/AgCl electrode was used as a reference electrode, a platinum wire as a counter electrode and a glassy graphite electrode as a working electrode. The sample to be analyzed was dissolved in a suitable solvent and, subsequently, it was deposited, with a calibrated capillary, on the working electrode, so as to form a film. The electrodes were immersed in a 0.1 M electrolytic solution of 95% tetrabutylammonium tetrafluoroborate in acetonitrile. The sample was subsequently subjected to a cyclic potential in the form of a triangular wave. At the same time, according to the applied potential difference, the current was monitored, which signals the occurrence of oxidation or reduction reactions of the species present.

The oxidation process corresponds to the removal of an electron from HOMO, while the reduction cycle corresponds to the introduction of an electron into the LUMO. The potentials for the formation of the radical cation and of the radical anion were obtained from the value of the peak onset (Eₒₙₛₑₜ), which is determined by molecules and/or chain segments with (E_{HOMO})-(E_{LUMO}) energy levels closer to the edges of the bands. The electrochemical potentials and those relating to the electronic levels can be correlated if both refer to the vacuum. For this purpose, the potential of ferrocene in vacuum, known in the literature and equal to −4.8 eV, was taken as a reference. The inter-solvent redox ferrocene/ferrocinium pair (Fc/Fc⁺) was selected because it has an oxidation-reduction potential independent of the working solvent.

The general formula for calculating the energies of the (E_{HOMO})-(E_{LUMO}) energy levels is therefore given by the following equation:

E (eV)=−4.8+[E_{1/2 Ag/AgCl}(Fc/Fc⁺)−E_{onset Ag/AgCl} (compound)]

wherein:
E=HOMO (E_{HOMO}) or LUMO (E_{LUMO}) energy level depending on the Eₒₙₛₑₜ value entered;
E_{1/2 Ag/AgCl}=half-wave potential of the peak corresponding to the redox ferrocene/ferrocinium pair (Fc/Fc⁺) measured under the same analysis conditions of the sample and with the same set of three electrodes used for the sample;
E_{onset Ag/AgCl}=onset potential measured for the compound in the anodic zone (oxidation) when you want to calculate the HOMO (E_{HOMO}) energy level and in the cathodic zone (reduction) when you want to calculate the LUMO (E_{LUMO}) energy level.

### EXAMPLE 1

### Synthesis of Compound GS7: 2,2′-(5,6-bis(4-(carbo-2-octyldecyloxy)-phenoxy)-benzo[c][1,2,5]thiadiazole-4,7-di-2-thienothiophene-5-diyl-bis-(methanylylidene)-3-oxo-2,3-dihydro-1H-indene-2,1-diylidene)dimalononitrile

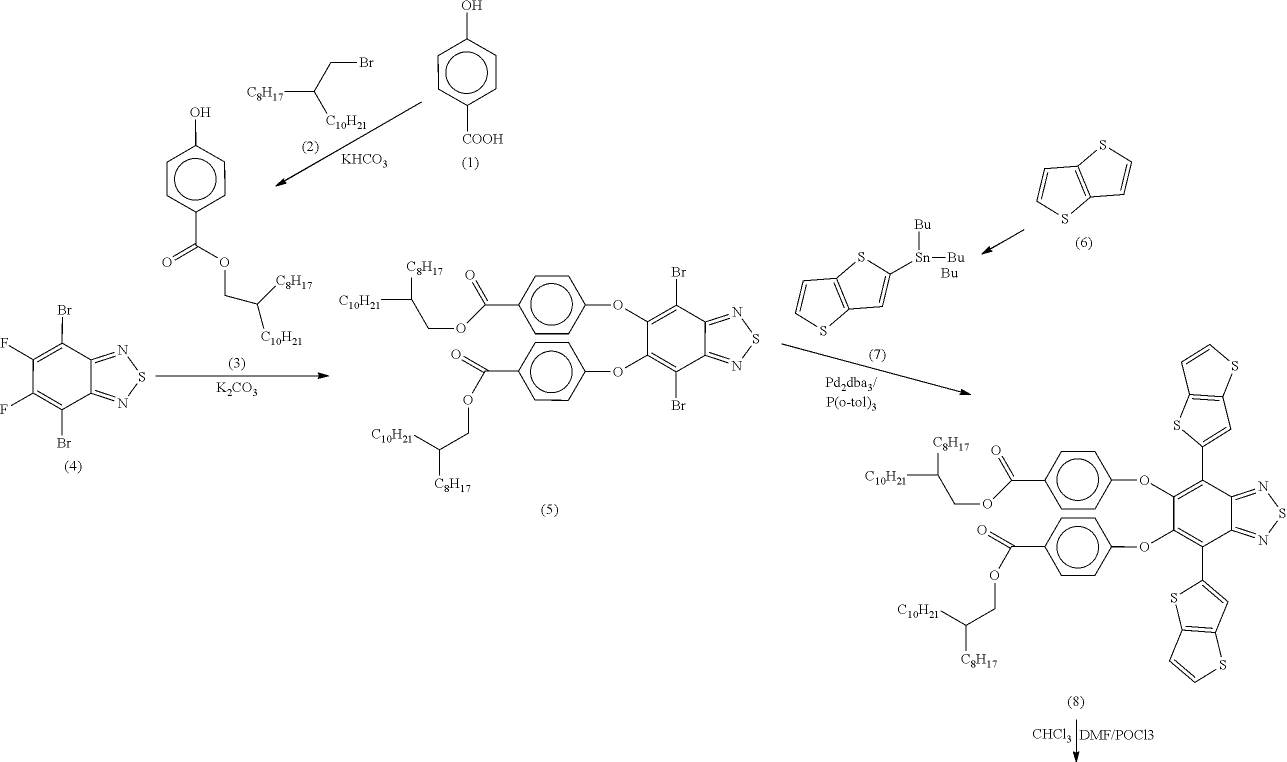

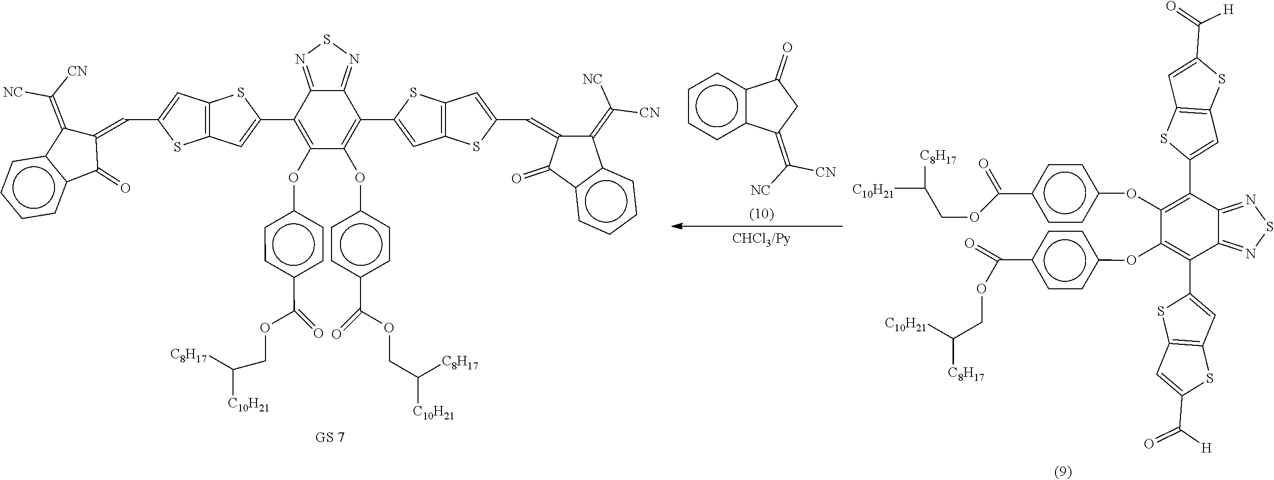

### Synthesis of 2-octyldodecyl 4-hydroxybenzoate (3)

The following were loaded into a 50 ml microwaveable vial: 4-hydroxybenzoic acid (1) (Merck) (1.5 g; 10.86 mmol), 2-octyldodecylbromide (2) (Merck) (3.9 g; 10.86 mmol), anhydrous N,N-dimethylformamide (Merck) (30 ml), potassium bicarbonate (Merck) (KHCO₃) (1.08 g; 10.86 mmol) and potassium iodide (Merck) (180.6 mg; 1.08 mmol): after insufflation with argon, the vial was placed in the microwave (Discover SP-D—CEM Corp.). After 1 hour, at 80° C., under stirring (medium stirring), the reaction mixture was poured into distilled water (50 ml) and extracted with ethyl ether (Merck) (3×30 ml). The organic phase (obtained by combining the three organic phases) was washed to neutral with distilled water (3×20 ml) and dried over sodium sulphate (Merck). The solvent was removed by distillation at reduced pressure and the residue obtained was purified by elution on a silica gel chromatographic column (eluent: n-heptane (Merck)/eluent 1 in gradient from 95/5 to 90/10 to 80/20, wherein the eluent 1 consist of a mixture of dichloromethane (Merck):ethyl acetate (Merck) in the ratio 1:1 v/v), obtaining 3.0 g (7.1 moles) of 2-octyldodecyl 4-hydroxybenzoate (3) (yield=66%).

### Synthesis of 4,7-dibromo-5,6-di(4-carbo(2-octyldodecyloxy)phenoxy)benzo-thiadiazole (5)

2-octyldodecyl 4-hydroxybenzoate (3) (2.17 g; 5.2 mmol) obtained as described above and potassium carbonate (K₂CO₃) (Merck) (717.0 mg; 5.2 mmol) were added to a solution of 4,7-dibromo-5,6-difluorobenzothiadiazole (4) (Merck) (800.0 mg; 2.4 mmol) in N,N-dimethylformamide (anhydrous) (Merck) (15 ml) in a 100 ml flask, equipped with magnetic stirring, thermometer and cooler, in an inert atmosphere: the mixture obtained was heated to 82° C. and kept, under stirring, at said temperature, for 12 hours. Subsequently, the reaction mixture was poured into distilled water (50 ml) and was extracted with ethyl ether (Merck) (3×30 ml). The organic phase (obtained by combining the three organic phases) was washed to neutral with distilled water (3×30 ml) and dried over sodium sulphate (Merck). The solvent was removed by distillation at reduced pressure and the residue obtained was purified by elution on a silica gel chromatographic column (eluent: n-heptane (Merck)/dichloromethane (Merck) in a gradient from 95/5 to 90/10 to 80/20) obtaining 2.6 g (2.3 mmoles) of 4,7-dibromo-5,6-di(4-carbo(2-octyldodecyloxy)phenoxy)benzothiadiazole (5) (yield=96%).

### Synthesis of 2-tributylstannylthienothiophene (7)

n-Butyllithium [1.6 M solution in hexane (Merck)](1.78 ml; 2.86 mmol) was added dropwise to a 0.12 M solution of 2,6-thienothiophene (6) (Merck) (0.36 g; 2.6 mmol) in anhydrous tetrahydrofuran (Merck) (22 ml) in a 100 ml flask, equipped with magnetic stirring, thermometer and cooler, placed in a dry ice bath at −78° C., in an inert atmosphere (argon): the reaction mixture obtained was kept under stirring, and the temperature was allowed to rise spontaneously to −50° C., in 3 hours. Subsequently, after bringing the temperature back to −78° C., tri-n-butylstannylchloride (Merck) (1.0 g; 0.85 ml; 3.12 mmol) was added dropwise: after 15 minutes the flask was removed from the dry ice bath, the temperature was allowed to rise spontaneously to 20° C. and the reaction mixture was kept, at said temperature, under stirring, for 12 hours. Subsequently, after adding a saturated aqueous solution of sodium bicarbonate (Merck) (20 ml), the reaction mixture was extracted with diethyl ether (Merck) (3×25 ml). The organic phase (obtained by combining the three organic phases) was washed with a saturated aqueous solution of sodium bicarbonate (Merck) (1×30 ml) and dried over sodium sulphate (Merck). The solvent was removed by distillation under reduced pressure obtaining 2-tri-n-butylstannylthienothiophene (7) which is used as such in the subsequent reaction.

### Synthesis of 4,7-di(2-thienothienyl)-5,6-di(4-carbo(2-octyldodecyloxy)phenoxy)-benzothiadiazole (8)

4,7-dibromo-5,6-di(4-carbo(2-octyldodecyloxy)phenoxy)benzothiadiazole (5) (1.1 g; 0.98 mmol) obtained as described above was added to a solution of 2-tri-n-butylstannylthienothiophene (7) (2.6 mmol) obtained as described above in anhydrous toluene (Merck) (20 ml) in a 100 ml flask, equipped with magnetic stirring, thermometer and cooler, under inert atmosphere. After removing the air present through 3 vacuum/nitrogen cycles, tris-dibenzylideneacetone dipalladium (Pd₂dba₃) (Merck) (21.0 mg; 0.02 mmol) and tris-o-tolylphosphine [P(o-tol)₃](Merck) (27.8 mg; 0.09 mmoles) were added: the mixture obtained was heated to 108° C. and kept under stirring at said temperature for 12 hours. Subsequently, after adding distilled water (50 ml), the reaction mixture was extracted with ethyl acetate (Merck) (3×50 ml). The organic phase (obtained by combining the three organic phases) was washed to neutral with distilled water (3×50 ml) and dried over sodium sulphate (Merck). After removing the solvent by distillation under reduced pressure, the residue obtained was purified by elution on a silica gel chromatographic column (eluent: n-heptane (Merck)/eluent 1 in the ratio 9/1 v/v, wherein the eluent 1 consists of a mixture of dichloromethane (Merck)/ethyl acetate (Merck) in the ratio 1/1 v/v), obtaining 1.1 g (0.88 mmol) of 4,7-di(2-thienothienyl)-5,6-di(4-carbo(2-octyldodedecyloxy)phenoxy)benzothiadiazole (8) (yield=90%).

### Synthesis of 4,7-di(2-(5-formylthienothienyl)-5,6-di(4-carbo(2-octyldodecyloxy)phenoxy)benzothiadiazole 9

N,N-dimethylformamide (DMF) (Merck) (2.7 ml) and, dropwise, phosphorus oxychloride (POCl₃) (Merck) (2.1 ml; 3.44 g; 22.4 mmol) were added to a solution of 4,7-di(2-thienothienyl)-5,6-di(4-carbo(2-octyldodecyloxy)phenoxy)benzothiadiazole (8) (1.4 g; 0.84 mmol) obtained as described above in anhydrous chloroform (CHCl₃) (Merck) (30.0 ml) in a 100 ml flask, equipped with magnetic stirring, thermometer and cooler, under an inert atmosphere, at 0° C.: the reaction mixture was placed under stirring and, after 30 minutes, it was heated to 69° C. and kept under stirring at said temperature for 48 hours. Subsequently, after adding a 10% solution of potassium acetate in water (20 ml), the reaction mixture was kept, under stirring, at 69° C., for 1 hour and subsequently extracted with ethyl acetate (Merck) (3×30 ml). The organic phase (obtained by combining the three organic phases) was washed to neutral with distilled water (3×30 ml) and dried over sodium sulphate (Merck). After removing the solvent by distillation under reduced pressure, the residue obtained was purified by elution on a silica gel chromatographic column (eluent n-heptane (Merck)/eluent 1 in gradient from 95/5 to 9/1 to 85/15 to 8/2, wherein the eluent 1 consists of a mixture of dichloromethane (Merck)/ethyl acetate (Merck) in the ratio 1/1 v/v), obtaining 910.0 mg (0.7 mmoles) of 4,7-di(2-(5-formylthienothienyl)-5,6-di(4-octyldodecyloxy)benzothiadiazole (9) (yield 83.3%).

### Synthesis of Compound GS7: 2,2′-(5,6-bis(4-(carbo-2-octyldecyloxy)-phenoxy)-benzo[c][1,2,5]thiadiazole-4,7-di-2-thienothiophene-5-diyl-bis (methanlylidene)-3-oxo-2,3-dihydro-1H-indene-2,1-diylidene)dimalononitrile

3-(dicyanomethylidene)indan-1-one (10) (Merck) (60.0 mg; 0.308 mmol) was added to a solution of 4,7-di(2-(5-formylthienothienyl)-5,6-di(4-octyldodecyloxyphenoxy)benzothiadiazole (9) (100.3 mg; 0.077 mmol) obtained as described above, in anhydrous chloroform (CHCl₃) (Merck) (22.5 ml) in a 100 ml flask, equipped with magnetic stirring, thermometer and cooler, in an inert atmosphere (argon): after removing the air from the reaction environment, by means of 3 vacuum/argon cycles, anhydrous pyridine (Py) (Merck) (500 μl) was added. After removing, again, the air from the reaction environment by 3 vacuum/argon cycles, the reaction mixture was heated to 69° C. and kept at said temperature, for 12 hours, under stirring. Subsequently, the temperature was allowed to drop spontaneously to 20° C. and ethanol (Merck) (15 ml) was added: the reaction mixture was kept at said temperature, for 20 minutes, under stirring. Subsequently, most of the organic solvent was removed by distillation under reduced pressure and the remaining residue was taken up with dichloromethane (Merck) (10 ml): the mixture obtained was added, dropwise, to ethanol (Merck) (20 ml). The precipitate obtained was isolated by filtration, washed with ethanol (Merck) (5×10 ml), acetonitrile (Merck) (1×10 ml) and, finally, with ethyl ether (Merck) (1×10 ml) obtaining 100 mg (0.060 mmol) of compound GS7 (yield=78%).

The compound GS7 was subjected to ¹H NMR characterization by operating as described above.

¹H-NMR (400 MHz, Chloroform-d) δ 8.99 (s, 2H), 8.88 (s, 2H), 8.69 (m, 2H), 8.06 (d, J=8.4 Hz, 4H), 8.02 (s, 2H), 7.96 (m, 2H), 7.79 (m, 4H), 6.85 (d, J=8.4 Hz, 4H), 4.24 (d, J=5.6 Hz, 4H), 1.77 (m, 2H), 1.48-1.17 (m, 32H), 0.87 (m, 12H).

The compound GS7 was also subjected to the other characterizations reported above: the absorption spectrum, the optical energy gap (E_{g}°), the values of the energy levels HOMO (E_{HOMO}), LUMO (E_{LUMO}) and the electrochemical band-gap (E_{gapEC}) have been acquired: the values obtained are reported in Table 2 and Table 3.

In Table 2 are reported, in order: the compound (Compound), the solvent used (Solvent), the value of the optical energy gap (E_{g}°), expressed in (eV), the maximum value of the lowest energy band in the absorption spectrum [λₘₐₓ (abs.)] expressed in (nm).

In Table 3 are reported, in order: the compound (Compound), the value of the HOMO (E_{HOMO}) energy level expressed in (eV), the value of the LUMO (E_{LUMO}) energy level expressed in (eV) and, finally the value of the electrochemical band-gap (E_{gapEC}) expressed in (eV).

FIG. 2 shows [the potential (E) measured in volts (V) vs ferrocene/ferrocinium (Fc/Fc⁺) is reported on the abscissa and the current density (i) measured in amperes (A) is reported on the ordinate)] the cyclic voltagram obtained by operating as described above.

### EXAMPLE 2

### Synthesis of Compound GS8: 2,2′-(((5,6-di(4-carbo(2-octyldodecyloxy)-phenoxy)benzo[c][1,2,5]thiadiazole-4,7-di-2-thienothiophene-5-diyl-bis(methanylidene)-bis-5,6-difluoro-3-oxo-2,3-dihydro-1H-indene-2,1-diylidene))dimalononitrile

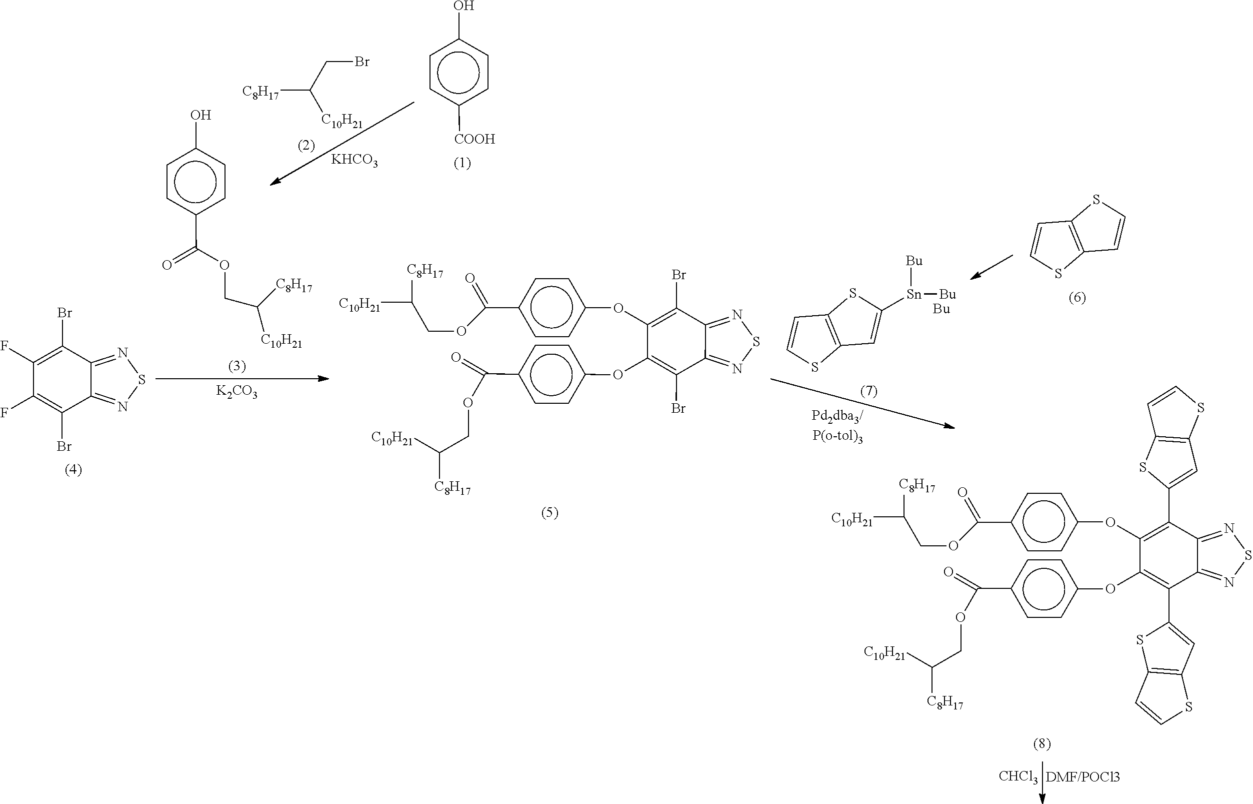

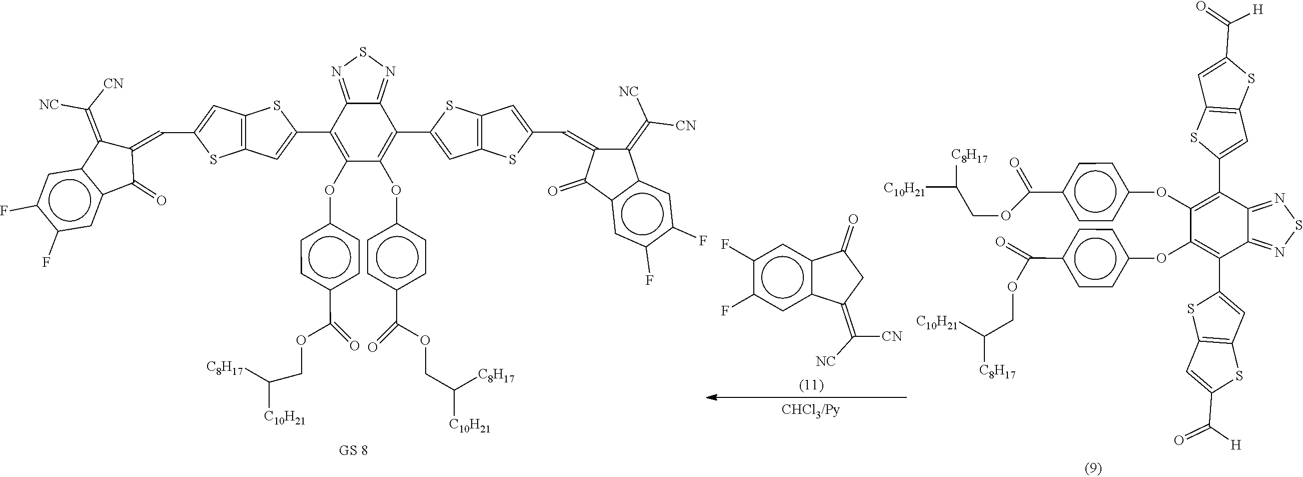

Anhydrous pyridine (Merck) (731 μl) was added to a solution of 4,7-di(2-(5-formylthienothienyl)-5,6-di(4-octyldodecyloxyphenoxy)benzothiadiazole (9) (146.5 mg; 0.11 mmoles) obtained as described above, in anhydrous chloroform (Merck) (32 ml) in a 100 ml flask, equipped with magnetic stirring, thermometer and cooler, in an inert atmosphere (argon). After removing the air from the reaction environment by 3 vacuum/argon cycles, the flask was placed in an ethanol and dry ice bath, the reaction mixture was cooled to −10° C., and added, dropwise, in 10 minutes, a solution, previously deaerated, by means of 3 vacuum/argon cycles, of 5,6-difluoro-3-(dicyanomethylidene)indan-1-one (11) (Merck) (104.0 mg; 0.45 mmol), in anhydrous chloroform (Merck) (10 ml). After removing the air from the reaction environment by means of 3 vacuum/argon cycles, the temperature was allowed to rise spontaneously to 0° C. and the reaction mixture was kept at said temperature, under stirring, for 10 minutes. Subsequently, the reaction mixture was heated to 65° C. and maintained, at said temperature, under stirring for 18 hours. Subsequently, the temperature was allowed to drop spontaneously to 20° C. and acetonitrile (Merck) (20 ml) was added: the reaction mixture was kept, at said temperature, under stirring, for 1 hour. Subsequently, most of the organic solvent was removed by distillation at reduced pressure and the remaining residue was taken up with chloroform (Merck) (10 ml): the mixture obtained was added, dropwise, to acetonitrile (Merck) (20 ml). The precipitate obtained was isolated by filtration, washed with acetonitrile (Merck) (5×10 ml), ethanol (Merck) (1×3 ml) and, finally, with ethyl ether (Merck) (1×10 ml) obtaining 142.2 mg (0.082 mmol) of compound GS8 (yield=75%). Compound GS8 was subjected to ¹H NMR characterization by operating as described above.

¹H-NMR (400 MHz, Chloroform-d) δ 9.08 (s, 2H), 8.93 (s, 2H), 8.58 (dd, J (H−F)=9.8, 6.3 Hz, 2H), 8.09 (s, 2H), 8.04 (d, J=8.3 Hz, 4H), 7.75 (t, J (H−F)=7.4 Hz, 2H), 6.81 (d, J=8.4 Hz, 4H), 4.23 (d, J=5.6 Hz, 4H), 1.76 (m, 2H), 1.45-1.20 (m, 32H), 0.89 (m, 12H)

The compound GS8 was also subjected to the other characterizations described above: the absorption spectrum, the optical energy gap (E_{g}°), the values of the energy levels HOMO (E_{HOMO}), LUMO (E_{LUMO}) and the electrochemical band-gap (E_{gapEC}) have been acquired: the values obtained are reported in Table 2 and Table 3.

In Table 2 are reported, in order: the compound (Compound), the solvent used (Solvent), the value of the optical energy gap (E_{g}°), expressed in (eV), the maximum value of the lowest energy band in the absorption spectrum [λₘₐₓ (abs.)] expressed in (nm).

In Table 3 are reported, in order: the compound (Compound), the value of the HOMO (E_{HOMO}) energy level expressed in (eV), the value of the LUMO (E_{LUMO}) energy level expressed in (eV) and, finally the value of the electrochemical band-gap (E_{gapEC}) expressed in (eV).

FIG. 3 shows [the potential (E) measured in volts (V) vs ferrocene/ferrocinium (Fc/Fc⁺) is reported on the abscissa and the current density (i) measured in amperes (A) is reported on the ordinate)] the cyclic voltagram obtained by operating as described above.

### EXAMPLE 3

### Synthesis of the Compound GS12: 2,2′-(5,6-bis(2-(carbo-2-octyldodecyloxy)phenoxy)benzo[c][1,2,5]thiadiazole-4,7-di-2-thienothiophene-5-diyl-bis(methanylylidene)-3-oxo-2,3-dihydro-1H-indene-2,1-diylidene)-dimalononitrile

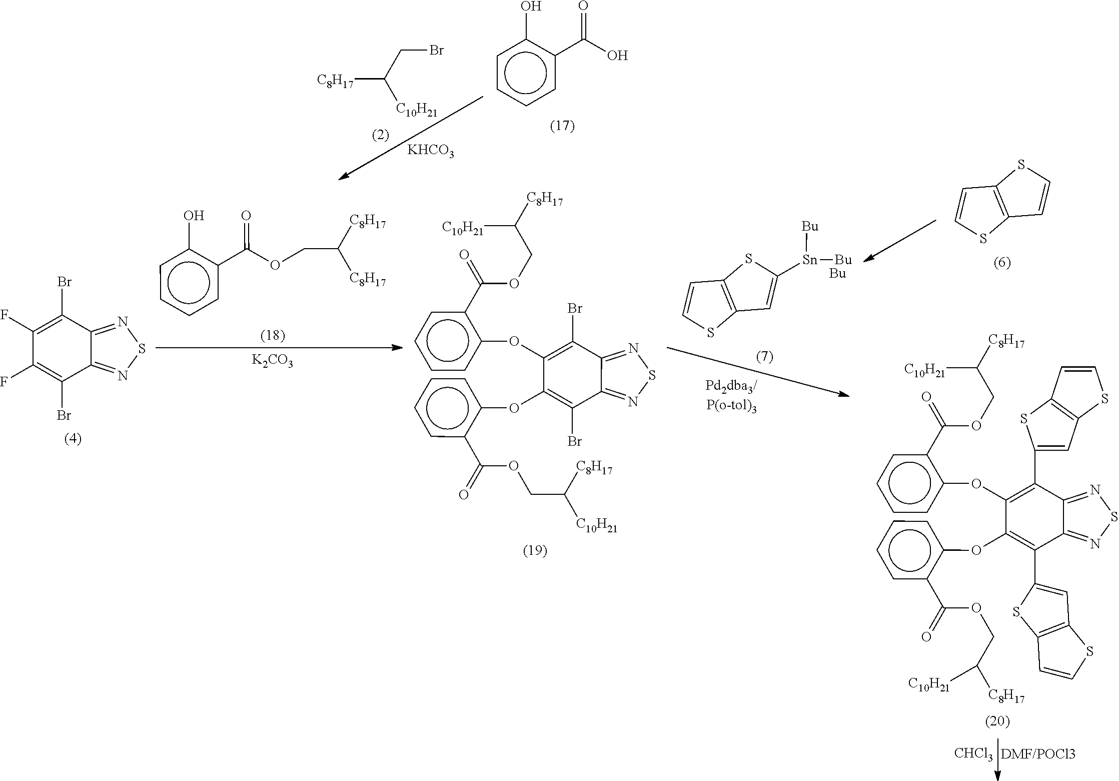

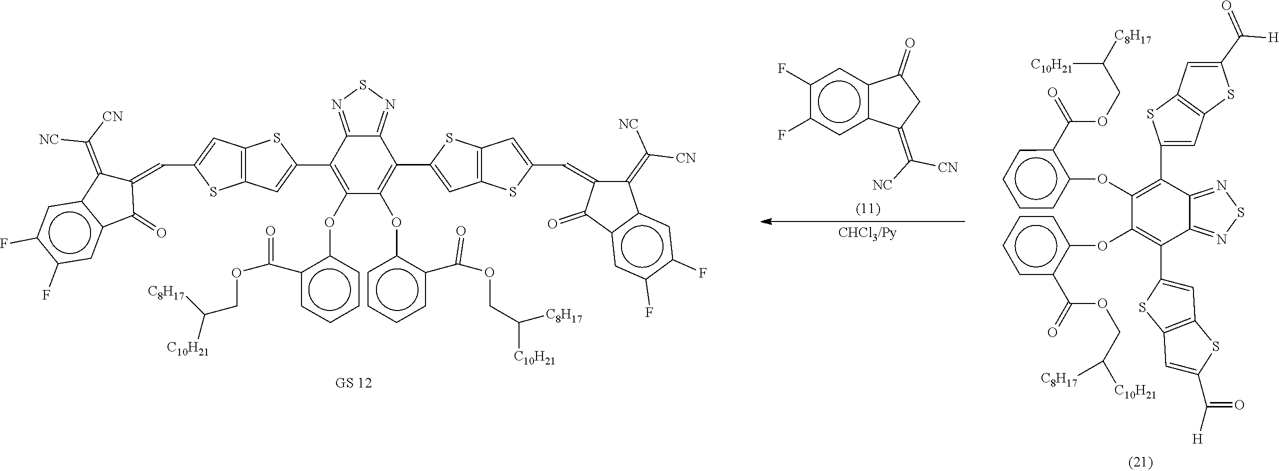

### Synthesis of 2-octyldodecyl 2-hydroxybenzoate (18)

N,N-dimethylformamide anhydrous (Merck) (30 ml), 2-octyldodecylbromide (2) (Merck) (3.9 g; 10.86 mmol), potassium bicarbonate (KHCO₃) (Merck) (1.08 g; 10.86 mmol) and potassium iodide (Merck) (180.6 mg; 1.08 mmol) were added to salicylic acid (17) (Merck) (1.5 g; 10.86 mmol) in a 100 ml flask, equipped with magnetic stirring, thermometer and cooler, in an inert atmosphere: the reaction mixture was heated to 82° C. and kept at said temperature, under stirring, for 16 hours. Subsequently, after adding distilled water (50 ml) and a 1 M hydrochloric acid solution (Merck) in order to bring the whole to pH 3, the reaction mixture was extracted with ethyl acetate (3×50 ml). The organic phase (obtained by combining the three organic phases) was washed to neutral with distilled water (3×30 ml) and dried over sodium sulphate (Merck). After removing the solvent by distillation under reduced pressure, the residue obtained was purified by elution on a silica gel chromatographic column (eluent n-heptane (Merck)/eluent 1 in gradient from 95/5 to 9/1 to 90/10 to 80/20, wherein the eluent 1 consists of a mixture of dichloromethane (Merck)/ethyl acetate (Merck) in the ratio 1/1 v/v), obtaining 4.0 g of 2-octyldodecyl 2-hydroxybenzoate (18) (yield=89%).

### Synthesis of 4,7-dibromo-5,6-di(2-carbo(2-octyldodecyloxy)phenoxy)benzo-thiadiazole (19)

2-octyldodecyl 2-hydroxybenzoate (18) (2.5 g; 6.1 mmol) obtained as described above and potassium carbonate (K₂CO₃) (Merck) (842.0 mg; 6.1 mmol) were added to a solution of 4,7-dibromo-5,6-difluorobenzothiadiazole (4) (Merck) (930.3 mg; 2.8 mmol) in anhydrous N,N-dimethylformamide (Merck) (18 ml) in a 100 ml flask, equipped with magnetic stirring, thermometer and cooler, in an inert atmosphere: the reaction mixture was heated to 82° C. and kept at said temperature, under stirring, for 12 hours. Subsequently, after adding distilled water (50 ml), the reaction mixture was extracted with ethyl acetate (Merck) (3×50 ml). The organic phase (obtained by combining the three organic phases) was washed to neutral with distilled water (3×50 ml) and dried over sodium sulphate (Merck). After removing the solvent by distillation under reduced pressure, the residue obtained was purified by elution on a silica gel chromatographic column (eluent: n-heptane (Merck)/dichloromethane (Merck) in a gradient from 95/5 to 90/10 to 80/20) to obtain 3.04 g (2.7 mmoles) of 4,7-dibromo-5,6-di(2-carbo(2-octyldodecyloxy)phenoxy)benzothiadiazole (19) (yield=96%).

### Synthesis of 4,7-di(2-thienothienyl)-5,6-di(2-carbo(2-octydodecyloxy)phenoxy)benzo-thiadiazole (20)

4,7-dibromo-5,6-di(2-carbo(2-octyldodecyloxy)phenoxy)benzothiadiazole (19) (1.1 g; 0.98 mmol) obtained as described above was added to a solution of 2-tri-n-butylstannylthienothiophene (7) (2.6 mmol) obtained as described above, in anhydrous toluene (Merck) (20 ml) in a 100 ml flask, equipped with magnetic stirring, thermometer and cooler, under inert atmosphere (argon). After removing the air present through 3 vacuum/nitrogen cycles, tris-dibenzylideneacetone dipalladium (Pd₂dba₃) (Merck) (21.0 mg; 0.02 mmol) and tris-o-tolylphosphine [P(o-tol)₃](Merck) (27.8 mg; 0.09 mmoles) were added: the mixture obtained was heated to 108° C. and kept under stirring at said temperature for 12 hours. Subsequently, after adding distilled water (50 ml), the reaction mixture was extracted with ethyl acetate (Merck) (3×50 ml). The organic phase (obtained by combining the three organic phases) was washed to neutral with distilled water (3×50 ml) and dried over sodium sulphate (Merck). After removing the solvent by distillation under reduced pressure, the residue obtained was purified by elution on a silica gel chromatographic column (eluent: n-heptane (Merck)/eluent 1 in the ratio 9/1 v/v, wherein the eluent 1 consists of a mixture of dichloromethane (Merck)/ethyl acetate (Merck) in the ratio 1/1 v/v), obtaining 1.1 g (0.88 mmol) of 4,7-di(2-thienothienyl)-5,6-di(2-carbo(2-octyldodecyloxy)phenoxy)benzothiadiazole (20) (yield=90%).

### Synthesis of 4,7-di(2-(5-formylthienothienyl)-5,6-di(2-carbo(2-octyldecyloxy) phenoxy)benzothiadiazole (21)

N,N-dimethylformamide (2.1 ml) and, dropwise, phosphorus oxychloride (POCl₃) (Merck) (1.7 ml; 2.8 g; 18.3 mmol), were added to a solution of 4,7-di(2-thienothienyl)-5,6-di(2-carbo(2-octyldodecyloxy)phenoxy)benzothiadiazole (20) (817.0 mg; 0.66 mmol) obtained as described above, in anhydrous chloroform (CHCl₃) (Merck) (25.0 ml) in a 100 ml flask, equipped with magnetic stirring, thermometer and cooler, under an inert atmosphere (argon), at 0° C.: the mixture obtained was stirred and, after 30 minutes, was heated to 69° C. and kept, under stirring, at said temperature, for 48 hours. Subsequently, after adding a 10% solution of potassium acetate (Merck) in water, the reaction mixture was kept, under stirring, at 69° C., for 1 hour and subsequently extracted with ethyl acetate (3×50 ml). The organic phase (obtained by combining the three organic phases) was washed to neutral with distilled water (3×50 ml) and dried over sodium sulphate (Merck). After removing the solvent by distillation under reduced pressure, the residue obtained was purified by elution on a silica gel chromatographic column (eluent: n-heptane (Merck)/eluent 1 in gradient from 95/5 to 9/1 to 85/15 to 8/2, wherein the eluent 1 consists of a mixture of dichloromethane (Merck)/ethyl acetate (Merck) in the ratio 1/1 v/v), obtaining 643.5 mg (0.5 mmol) of 4,7-di(2-(5-formylthienothienyl)-5,6-di(2-carbo(2-octyldodecyloxy)-phenoxy)benzo-thiadiazole (21) (yield=75%).

### Synthesis of the Compound GS12: 2,2′-(5,6-bis(2-(carbo-2-octyldodecyloxy)phenoxy)benzo[c][1,2,5]thiadiazole-4,7-di-2-thienothiophene-5-diyl-bis(methanylylidene)-3-oxo-2,3-dihydro-1H-indene-2,1-diylidene)-dimalononitrile

Anhydrous pyridine (Py) (Merck) (935 μl) was added to a solution of 4,7-di(2-(5-formylthienothienyl)-5,6-di(4-octyldodecyloxyphenoxy)benzothiadiazole (21) (203, 6 mg; 0.16 mmol) obtained as described above, in anhydrous chloroform (CHCl₃) (Merck) (50 ml) in a 100 ml flask, equipped with magnetic stirring, thermometer and cooler, in an inert atmosphere (argon). After removing the air from the reaction environment by 3 vacuum/argon cycles, the flask was placed in an ethanol and dry ice bath, the reaction mixture was cooled to −10° C., and was added, dropwise, in 10 minutes, a previously deaerated solution, by means of 3 vacuum/argon cycles, of 5,6-difluoro-3-(dicyanomethylidene)indan-1-one (11) (Merck) (104.0 mg; 0.45 mmoles), in anhydrous chloroform (Merck) (10 ml). After removing the air from the reaction environment by 3 vacuum/argon cycles, the temperature was allowed to rise spontaneously to 20° C. and the mixture reaction was maintained, at this temperature, under stirring, for 10 minutes. Subsequently, the reaction mixture was heated to 65° C. and maintained, at said temperature, under stirring for 18 hours. Subsequently, the temperature was allowed to drop spontaneously to 20° C. and acetonitrile (Merck) (20 ml) was added: the reaction mixture was kept under stirring at said temperature for 1 hour. Subsequently, most of the organic solvent was removed by distillation at reduced pressure and the remaining residue was taken up with chloroform (Merck) (10 ml): the mixture obtained was added, dropwise, to acetonitrile (Merck) (20 ml). The precipitate obtained was isolated by filtration, washed with acetonitrile (Merck) (5×10 ml), ethanol (Merck) (1×3 ml) and, finally, with ethyl ether (Merck) (1×3 ml) obtaining 230, 0 mg (0.13 mmol) of compound GS12 (yield=83%).

The compound GS12 was subjected to ¹H NMR characterization by operating as described above.

¹H-NMR (400 MHz, Chloroform-d) δ 9.08 (s, 2H), 8.92 (s, 2H), 8.57 (dd, J (H−F)=9.8, 6.4 Hz, 2H), 8.09 (s, 2H), 7.95 (d, J=7.9 Hz, 2H), 7.73 (t, J(H−F)=7.5 Hz, 2H), 7.38 (t, J=7.9 Hz, 2H), 7.17 (t, J=7.6 Hz, 2H), 6.76 (d, J=8.4 Hz, 2H), 3.96 (dd, J=10.9, 5.6 Hz, 2H), 3.78 (dd, J=11.0, 6.5 Hz, 2H), 1.65 (m, 2H), 1.44-1.14 (m, 64H), 0.88 (m, 12H).

The compound GS12 was also subjected to the other characterizations reported above: the absorption spectrum, the optical energy gap (E_{g}°), the values of the energy levels HOMO (E_{HOMO}), LUMO (E_{LUMO}) and the electrochemical band-gap (E_{gapEC}) have been acquired: the values obtained are reported in Table 2 and Table 3.

In Table 2 are reported, in order: the compound (Compound), the solvent used (Solvent), the value of the optical energy gap (E_{g}°), expressed in (eV), the maximum value of the lowest energy band in the absorption spectrum [λₘₐₓ (abs.)] expressed in (nm).

In Table 3 are reported, in order: the compound (Compound), the value of the HOMO (E_{HOMO}) energy level expressed in (eV), the value of the LUMO (E_{LUMO}) energy level expressed in (eV) and, finally the value of the electrochemical band-gap (E_{gapEC}) expressed in (eV).

FIG. 4 shows [the potential (E) measured in volts (V) vs ferrocene/ferrocinium (Fc/Fc⁺) is shown on the abscissa and the current density (i) measured in amperes (A) is shown on the ordinate)] the cyclic voltagram obtained by operating as described above.

### EXAMPLE 4

### Synthesis of Compound GS13: 2,2′-(5,6-bis(3-(carbo-2-octyldodecyloxy)-phenoxy)benzo[c][1,2,5]thiadiazole-4,7-di-2-thienothiophene-5-diyl-bis-(methanylylidene)-3-oxo-2,3-dihydro-1H-indene-2,1-diylidene)dimalononitrile

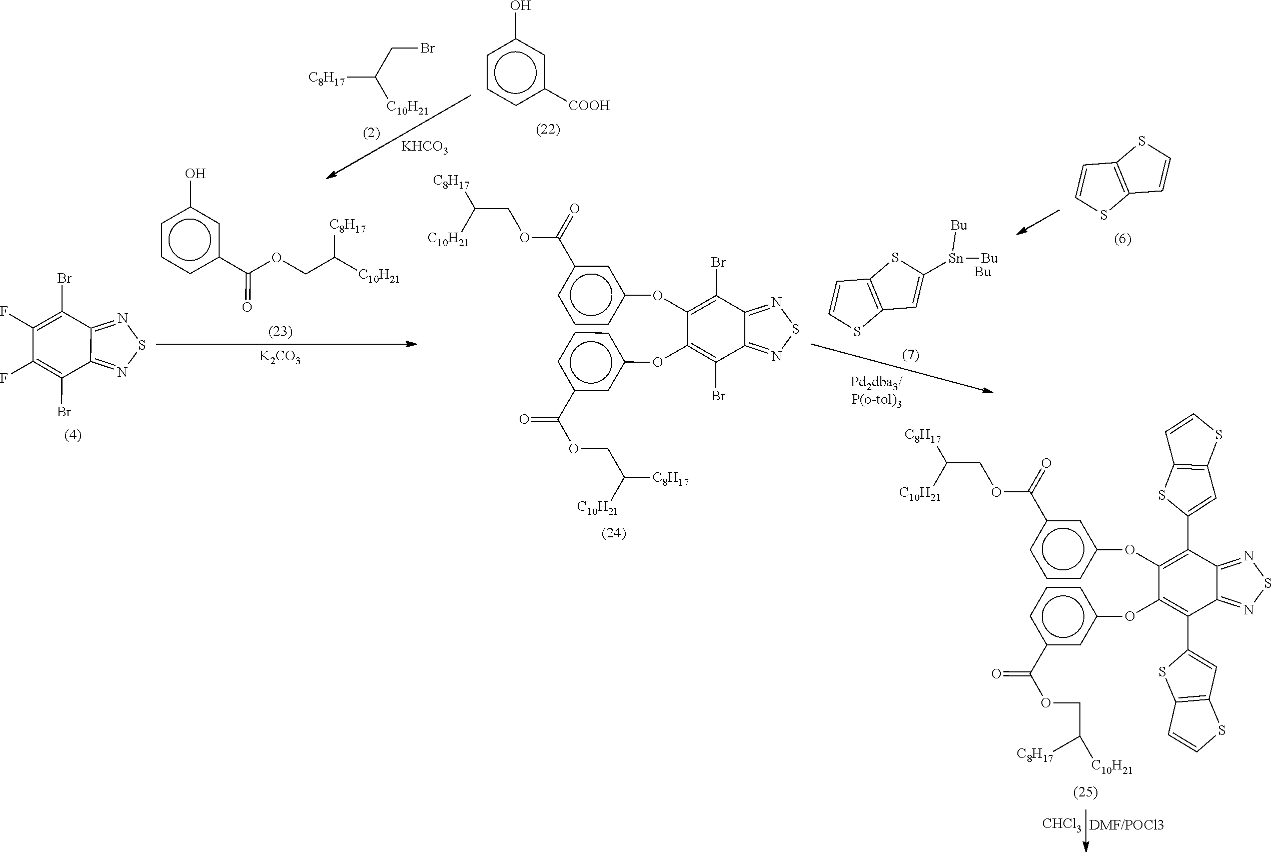

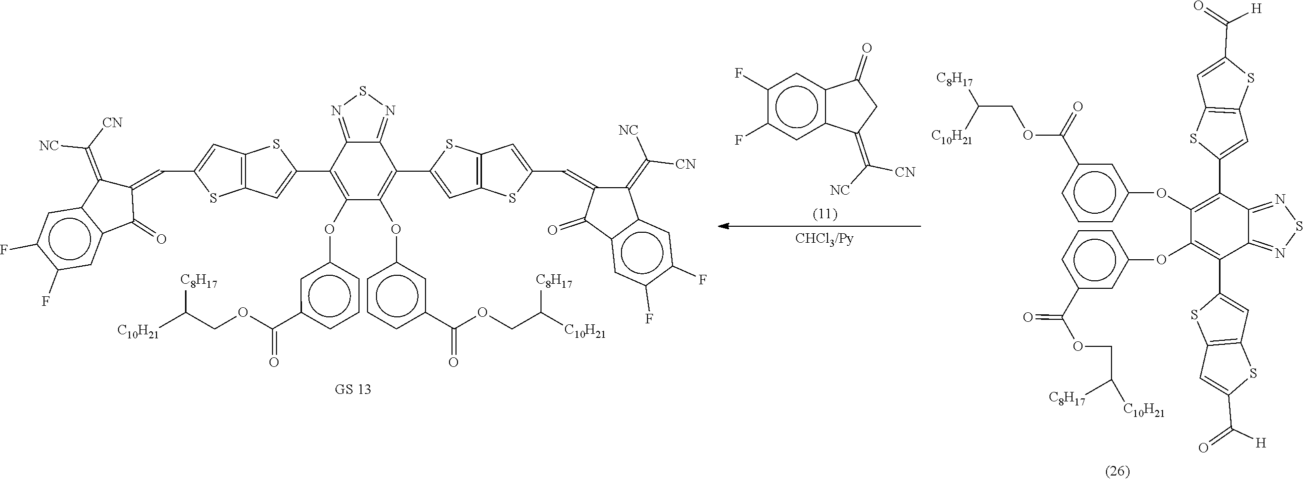

### Synthesis of 2-octyldodecyl 3-hydroxybenzoate (23)

N,N-dimethylformamide anhydrous (Merck) (30 ml), 2-octyldodecylbromide (Merck) (3.9 g; 10.86 mmol), potassium bicarbonate (KHCO₃) (Merck) (1.08 g; 10.86 mmol) and potassium iodide (Merck) (180.6 mg; 1.08 mmol) were added to 3-hydroxybenzoic acid (22) (Merck) (1.5 g; 10.86 mmol) in a 100 ml flask, equipped with magnetic stirring, thermometer and cooler, in an inert atmosphere (argon): the reaction mixture was heated to 82° C. and kept at said temperature, under stirring, for 16 hours. Subsequently, after adding distilled water (50 ml) and a 1 M hydrochloric acid solution (Merck) in order to bring the whole to pH 3, the reaction mixture was extracted with ethyl acetate (3×30 ml). The organic phase (obtained by combining the three organic phases) was washed to neutral with distilled water (3×30 ml) and dried over sodium sulphate (Merck). After removing the solvent by distillation under reduced pressure, the residue obtained was purified by elution on a silica gel chromatographic column (eluent n-heptane (Merck)/eluent 1 in gradient from 95/5 to 9/1 to 90/10 to 80/20, wherein the eluent 1 consists of a mixture of dichloromethane (Merck)/ethyl acetate (Merck) in the ratio 1/1 v/v), obtaining 4.0 g of 2-octyldodecyl 3-hydroxybenzoate (23) (yield=89%).

### Synthesis of 4,7-dibromo-5,6-di(3-carbo(2-octyldodecyloxy)phenoxy)benzo-thiadiazole (24)

2-octyldodecyl 3-hydroxybenzoate (23) (2.5 g; 6.1 mmol) obtained as described above and potassium carbonate (K₂CO₃) (Merck) (842.0 mg; 6.1 mmol) were added to a solution of 4,7-dibromo-5,6-difluorobenzothiadiazole (4) (Merck) (930.3 mg; 2.8 mmol) in anhydrous N,N-dimethylformamide (Merck) (18 ml) in a 100 ml flask, equipped with magnetic stirring, thermometer and cooler, in an inert atmosphere (argon) obtaining a reaction mixture which was heated to 82° C. and maintained, under stirring, at said temperature, for 12 hours. Subsequently, after adding distilled water (50 ml) the reaction mixture was extracted with ethyl ether (Merck) (3×50 ml). The organic phase (obtained by combining the three organic phases) was washed to neutral with distilled water (3×30 ml) and dried over sodium sulphate (Merck). After removing the solvent by distillation under reduced pressure, the residue obtained was purified by elution on a silica gel chromatographic column (eluent n-heptane (Merck)/dichloromethane (Merck) in gradient from 95/5 to 90/10 to 80/20, obtaining 4,7-dibromo-5,6-di(3-carbo(2-octyldodecyloxy)phenoxy)benzothiadiazole (24) (2.8 g; 2.5 mmol) (yield=88%).

### Synthesis of 4,7-di(2-thienothienyl)-5,6-di(3-carbo(2-octyldodecyloxy)phenoxy)benzo-thiadiazole (25)

4,7-dibromo-5,6-di(3-carbo(2-octyldodecyloxy)phenoxy)benzothiadiazole (24) (1.1 g; 0.98 mmol) obtained as described above was added to a solution of 2-tri-n-butylstannylthienothiophene (7) (2.6 mmol) obtained as described above, in anhydrous toluene (Merck) (20 ml) in a 100 ml flask, equipped with magnetic stirring, thermometer and cooler, under inert atmosphere. After removing the air present through 3 vacuum/nitrogen cycles, tris-dibenzylideneacetone dipalladium (Pd₂dba₃) (Merck) (21.0 mg; 0.02 mmol) and tris-o-tolylphosphine [P(o-tol)₃](Merck) (27.8 mg; 0.09 mmoles) were added: the mixture obtained was heated to 108° C. and kept under stirring at said temperature for 12 hours. Subsequently, after adding distilled water (50 ml), the reaction mixture was extracted with ethyl acetate (Merck) (3×50 ml). The organic phase (obtained by combining the three organic phases) was washed to neutral with distilled water (3×30 ml) and dried over sodium sulphate (Merck). After removing the solvent by distillation under reduced pressure, the residue obtained was purified by elution on a silica gel chromatographic column (eluent: n-heptane (Merck)/eluent 1 in the ratio 9/1 v/v, wherein the eluent 1 consists of a mixture of dichloromethane (Merck)/ethyl acetate (Merck) in the ratio 1/1 v/v), obtaining 0.9 g (0.72 mmol) of 4,7-di(2-thienothienyl)-5,6-di(3-carbo(2-octyldodecyloxy)phenoxy)benzothiadiazole (25) (yield=74%).

### Synthesis of 4,7-di(2-(5-formylthienothienyl)-5,6-di(3-carbo(2-octyldodecyloxy) phenoxy)-benzothiadiazole (26)

N,N-dimethylformamide (DMF) (Merck) (2.1 ml) and, dropwise, phosphorus oxychloride (POCl₃) (Merck) (1.7 ml; 2.8 g; 18.3 mmol) were added to a solution of 4,7-dithienothienyl-5,6-di(4-octyldodecyloxy-phenoxy)benzothiadiazole (25) (817.0 g; 0.66 mmol) obtained as described above in anhydrous chloroform (CHCl₃) (Merck) (30.0 ml) in a 100 ml flask, equipped with magnetic stirring, thermometer and cooler, in an inert atmosphere, at 0° C.: the mixture obtained was stirred and, after 30 minutes, was heated at 69° C. and maintained, under stirring, at said temperature for 48 hours. Subsequently, after adding a 10% solution of potassium acetate (Merck) in water, the reaction mixture was kept, under stirring, at 69° C., for 1 hour and subsequently extracted with ethyl acetate (Merck) (3×50 ml). The organic phase (obtained by combining the three organic phases) was washed to neutral with distilled water (3×30 ml) and dried over sodium sulphate (Merck). After removing the solvent by distillation under reduced pressure, the residue obtained was purified by elution on a silica gel chromatographic column (eluent n-heptane (Merck)/eluent 1 in gradient from 95/5 to 9/1 to 85/15 to 8/2, wherein the eluent 1 consists of a mixture of dichloromethane (Merck)/ethyl acetate (Merck) in the ratio 1/1 v/v), obtaining 600.0 mg (0.46 mmol) of 4,7-di(2-(5-formylthienothienyl)-5,6-di(3-carbo(2-octyldodecyloxy)phenoxy)benthiadiazole (26) (yield=70%).

### Synthesis of Compound GS13: 2,2′-(5,6-bis(3-(carbo-2-octyldodecyloxy)phenoxy)benzo[c][1,2,5]thiadiazole-4,7-di-2-thienothiophene-5-diyl-bis(methanylylidene)-3-oxo-2,3-dihydro-1H-indene-2,1-diylidene)-dimalononitrile

Anhydrous pyridine (Py) (Merck) (1 ml) was added to a solution of 4,7-di(2-(5-formylthienothienyl)-5,6-di(3-carbo(2-octyldodecyloxy)phenoxy)benzo-thiadiazole (26) (216, 8 mg; 0.17 mmol) obtained as described above in anhydrous chloroform (CHCl₃) (Merck) (50 ml) in a 100 ml flask, equipped with magnetic stirring, thermometer and cooler, in an inert atmosphere (argon). After removing the air from the reaction environment by 3 vacuum/argon cycles, the flask was placed in an ethanol and dry ice bath, the reaction mixture was cooled to −10° C., and a solution previously deaerated, by means of 3 vacuum/argon cycles, of 5,6-difluoro-3-(dicyanomethylidene)indan-1-one (11) (Merck) (104.0 mg; 0.45 mmoles) in anhydrous chloroform (Merck) (10 ml) was added dropwise, in 10 minutes. After removing the air from the reaction environment by 3 vacuum/argon cycles, the temperature was allowed to rise spontaneously to 20° C. and the reaction mixture were maintained at that temperature, under stirring, for 10 minutes. Subsequently, the reaction mixture was heated to 65° C. and maintained, at said temperature, under stirring for 18 hours. Subsequently, the temperature was allowed to drop spontaneously to 20° C. and acetonitrile (Merck) (20 ml) was added: the reaction mixture was kept under stirring at said temperature for 1 hour. Subsequently, most of the organic solvent was removed by distillation at reduced pressure and the remaining residue was taken up with chloroform (Merck) (10 ml): the mixture obtained was added, dropwise, to acetonitrile (Merck) (20 ml). The precipitate obtained was isolated by filtration, washed with acetonitrile (Merck) (5×10 ml), ethanol (Merck) (1×3 ml) and, finally, with ethyl ether (Merck) (1×3 ml) obtaining 220.0 mg (0.13 mmoles) of compound GS13 (yield=75%).

Compound GS13 was subjected to characterization of ¹H NMR operating as described above.

¹H-NMR (400 MHz, Chloroform-d) δ 9.05 (s, 2H), 8.89 (s, 2H), 8.55 (dd, J (H−F)=9.8, 6.4 Hz, 2H), 8.05 (s, 2H), 7.84 (dt, J=7.8, 1.2 Hz, 2H), 7.74 (t, J (H−F)=7.5 Hz, 2H), 7.44 (t, J=8.0 Hz, 2H), 7.38 (dd, J=2.8, 1.4 Hz, 2H), 6.96 (ddd, J=8.3, 2.8, 1.0 Hz, 2H), 4.18 (d, J=5.5 Hz, 4H), 1.72 (m, 2H), 1.50-1.14 (m, 64H), 0.90 (m, 12H).

The compound GS13 was also subjected to the other characterizations described above: the absorption spectrum, the optical energy gap (E_{g}°), the values of the energy levels HOMO (E_{HOMO}), LUMO (E_{LUMO}) and the electrochemical band-gap (E_{gapEC}) were acquired: the values obtained are reported in Table 2 and Table 3.

In Table 2 are reported, in order: the compound (Compound), the solvent used (Solvent), the value of the optical energy gap (E_{g}°), expressed in (eV), the maximum value of the lowest energy band in the absorption spectrum [λₘₐₓ (abs.)] expressed in (nm).

In Table 3 are reported, in order: the compound (Compound), the value of the HOMO (E_{HOMO}) energy level expressed in (eV), the value of the LUMO (E_{LUMO}) energy level expressed in (eV) and, finally the value of the electrochemical band-gap (E_{gapEC}) expressed in (eV).

FIG. 5 shows [the potential (E) measured in volts (V) vs ferrocene/ferrocinium (Fc/Fc⁺) is shown on the abscissa and the current density (i) measured in amperes (A) is shown on the ordinate)] the cyclic voltagram obtained by operating as described above.

### EXAMPLE 5

### Synthesis of the compound GS14: 2,2′-(5,6-bis(3,5-di(carbo-2-octyldodecyloxy)phenoxy)benzo[c][1,2,5]thiadiazole-4,7-di-2-thienothiophene-5-diyl-bis-(methanlylidene)-3-oxo-2,3-dihydro-1H-indene-2,1-diylidene)-dimalononitrile

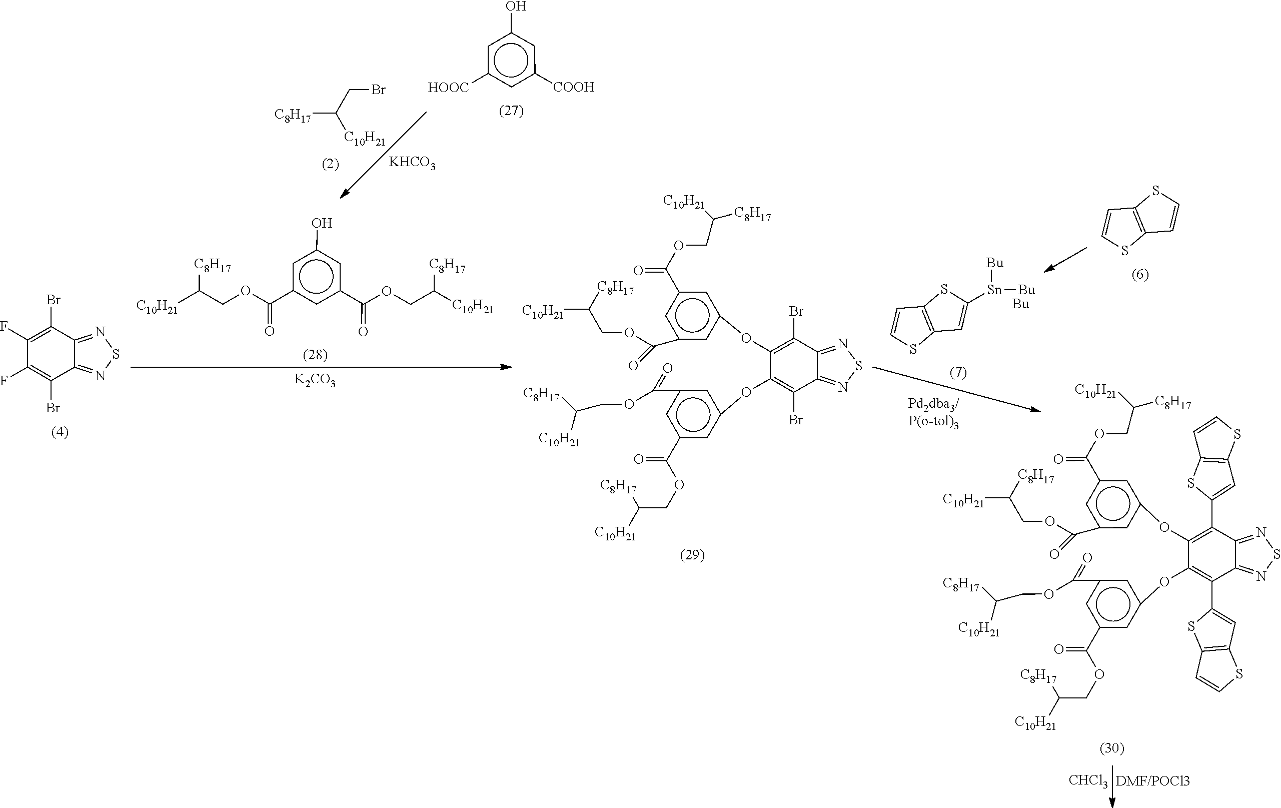

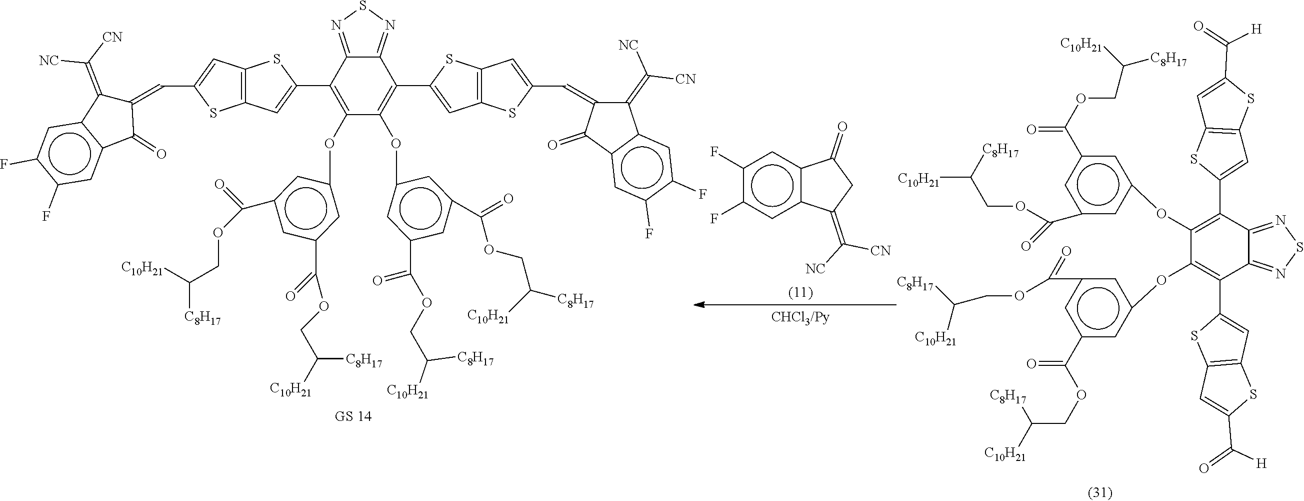

### Synthesis of 2-octyldodecyl 3-hydroxyisophthalate (28)

N,N-dimethylformamide anhydrous (Merck) (20 ml), 2-octyldodecylbromide (2) (Merck) (3.1 g; 8.54 mmol), potassium bicarbonate (KHCO₃) (Merck) (854, 5 mg; 8.54 mmol) and potassium iodide (Merck) (146.0 mg; 0.88 mmol) were added to 3-hydroxyisophthalic acid (27) (Merck) (776.7 mg; 4.3 mmol) in a 100 ml flask, equipped with magnetic stirring, thermometer and cooler, in an inert atmosphere: the reaction mixture was heated to 82° C. and kept at said temperature, under stirring, for 16 hours. Subsequently, after adding distilled water (50 ml) and a 1 M hydrochloric acid solution (Merck) in order to bring the whole to pH 3, the reaction mixture was extracted with ethyl acetate (Merck) (3×50 ml). The organic phase (obtained by combining the three organic phases) was washed to neutral with distilled water (3×30 ml) and dried over sodium sulphate (Merck). After removing the solvent by distillation at reduced pressure, the residue obtained was purified by elution on a silica gel chromatographic column (eluent n-heptane (Merck)/eluent 1 in gradient from 95/5 to 90/10, wherein the eluent 1 consists of a mixture of dichloromethane (Merck)/ethyl acetate (Merck) in the ratio 1/1 v/v), obtaining 635.0 mg (0.86 mmoles) of 2-octyldodecyl 3-hydroxyisophthalate (28) (yield=20%).

### Synthesis of 4,7-dibromo-5,6-di(3,5-di-carbo(2-octyldodecyloxy)phenoxy)-benzothiadiazole (29)

2-octyldodecyl 3-hydroxyisophthalate (28) (537.0 mg; 0.72 mmol) obtained as described above and potassium carbonate (K₂CO₃) (Merck) (109.0 mg; 0.79 mmol) were added to a solution of 4,7-dibromo-5,6-difluorobenzothiadiazole (4) (Merck) (113.0 mg; 0.34 mmol) in anhydrous N,N-dimethylformamide (Merck) (10 ml) in a 100 ml flask, equipped with magnetic stirring, thermometer and cooler, in an inert atmosphere: the reaction mixture was heated to 82° C. and kept at said temperature, under stirring, for 12 hours. Subsequently, after adding distilled water (30 ml), the reaction mixture was extracted with ethyl ether (Merck) (3×30 ml). The organic phase (obtained by combining the three organic phases) was washed to neutral with distilled water (3×30 ml) and dried over sodium sulphate (Merck). After removing the solvent by distillation under reduced pressure, the residue obtained was purified by elution on a silica gel chromatographic column (eluent n-heptane (Merck)/dichloromethane (Merck) in a gradient from 95/5 to 90/10 to 80/20, yielding 450.7 mg (0.25 mmoles) of 4,7-dibromo-5,6-di(3,5-di-carbo(2-octyldodecyloxy)phenoxy)benzothiadiazole (29) (yield=75%).

### Synthesis of 4,7-di(2-thienothienyl)-5,6-di(3,5-di-carbo(2-octyldodecyloxy)-phenoxy)benzothiadiazole (30)

4,7-dibromo-5,6-di(3,5-di-carbo(2-octyldodecyloxy)phenoxy)benzo-thiadiazole (29) (450.2 mg; 0.27 mmol) obtained as described above was added to a solution of 2-tri-n-butyl-stannylthienothiophene (7) (0.65 mmol) obtained as described above, in anhydrous toluene (Merck) (10 ml) in a 100 ml flask, equipped with magnetic stirring, thermometer and cooler in an inert atmosphere. After removing the air present through 3 vacuum/nitrogen cycles, tris-dibenzylideneacetone dipalladium (Pd₂dba₃) (Merck) (6.0 mg; 6.5×10−3 mmol) and tris-o-tolylphosphine were added [P(o-tol)₃](Merck) (8.0 mg; 0.026 mmoles) were added: the mixture obtained was heated to 108° C. and kept under stirring at said temperature for 12 hours. Subsequently, after adding distilled water (50 ml), the reaction mixture was extracted with ethyl acetate (Merck) (3×20 ml). The organic phase (obtained by combining the three organic phases) was washed to neutral with distilled water (3×30 ml) and dried over sodium sulphate (Merck). After removing the solvent by distillation under reduced pressure, the residue obtained was purified by elution on a silica gel chromatographic column (eluent: n-heptane (Merck)/eluent 1 in the ratio 9/1 v/v, wherein the eluent 1 consists of a mixture of dichloromethane (Merck)/ethyl acetate (Merck) in the ratio 1/1 v/v), obtaining 220.0 mg (0.72 mmol) of 4,7-di(2-thienothienyl)-5,6-di(3,5-di-carbo(2-octyldodecyloxy)phenoxy)benzothiadiazole (30) (yield=43%).

### Synthesis of 4,7-di(2-(5-formylthienothienyl)-5,6-di(3,5-di-carbo(2-octyldodecyloxy)phenoxy)benzothiadiazole (31)

N,N-dimethylformamide (DMF) (Merck) (0.5 ml) and, dropwise, phosphorus oxychloride (POCl₃) (Merck) (400.0 ml; 0.66 g; 4.3 mmol) were added to a solution of 4,7-di(2-thienothienyl)-5,6-di(3,5-di-carbo(2-octyldodecyloxy)phenoxy)benzothiadiazole (30) (215.0 mg; 0.11 mmol) obtained as described above, in anhydrous chloroform (CHCl₃) (Merck) (10.0 ml) in a 100 ml flask, equipped with magnetic stirring, thermometer and cooler, in an inert atmosphere, at 0° C.: the mixture obtained was placed under stirring and, after 30 minutes, was heated to 69° C. and kept under stirring at said temperature for 48 hours. Subsequently, after adding a 10% solution of potassium acetate in water, the reaction mixture was kept, under stirring, at 69° C., for 1 hour and subsequently extracted with ethyl acetate (Merck) (3×50 ml). The organic phase (obtained by combining the three organic phases) was washed to neutral with distilled water (3×30 ml) and dried over sodium sulphate (Merck). After removing the solvent by distillation under reduced pressure, the residue obtained was purified by elution on a silica gel chromatographic column (eluent n-heptane (Merck)/eluent 1 in gradient from 95/5 to 9/1 to 85/15 to 8/2, wherein the eluent 1 consists of a mixture of dichloromethane (Merck)/ethyl acetate (Merck) in the ratio 1/1 v/v), obtaining 121.0 mg (0.06 mmoles) of 4,7-di(2-(5-formylthienothienyl)-5,6-di(3,5-di-carbo(2-octyldodecyloxy)phenoxy)benzothiadiazole (31) (yield=56%).

### Synthesis of Compound GS14: 2,2′-(5,6-bis(3,5-di(carbo-2-octyldodecyloxy)phenoxy)benzo[c][1,2,5]thiadiazole-4,7-di-2-thienothiophene-5-diyl-bis-(methanylidene)-3-oxo-2,3-dihydro-1H-indene-2,1-diylidene)-dimalononitrile

Anhydrous pyridine (Py) (Merck) (371.2 μl) was added to a solution of 4,7-di(2-(5-formylthienothienyl)-5,6-di(3,5-di-carbo(2-octydecyloxy)phenoxy)-benzothiadiazole (31) (121.0 mg; 0.06 mmol) obtained as described above, in anhydrous chloroform (CHCl₃) (Merck) (20 ml) in a 100 ml flask, equipped with magnetic stirring, thermometer and cooler, in an inert atmosphere (argon). After removing the air from the reaction environment by 3 vacuum/argon cycles, the flask was placed in an ethanol and dry ice bath, the reaction mixture was cooled at −10° C., and a solution, previously deaerated, by means of 3 vacuum/argon cycles, of 5,6-difluoro-3-(dicyanomethylidene)indan-1-one (11) (Merck) (57.0 mg; 0.25 mmol) in anhydrous chloroform (Merck) (5 ml) was added dropwise in 10 minutes. After removing the air from the reaction environment by 3 vacuum/argon cycles, the temperature was allowed to rise spontaneously at 20° C. and the reaction mixture was maintained at said temperature, under stirring, for 10 minutes. Subsequently, the reaction mixture was heated to 65° C. and maintained, at said temperature, under stirring for 18 hours. Subsequently, the temperature was allowed to drop spontaneously to 20° C. and acetonitrile (Merck) (10 ml) was added: the reaction mixture was kept, at said temperature, under stirring, for 1 hour. Subsequently, most of the organic solvent was removed by distillation at reduced pressure and the remaining residue was taken up with chloroform (Merck) (5 ml): the mixture obtained was added, dropwise, to acetonitrile (Merck) (10 ml). The precipitate obtained was isolated by filtration, washed with acetonitrile (Merck) (5×10 ml), ethanol (Merck) (1×2 ml)) and, finally, with ethyl ether (Merck) (1×2 ml) to obtain 100.0 mg (0.042 mmoles) of the product GS14 (yield=68%).

Compound GS14 was subjected to ¹H NMR characterization by operating as described above.

¹H-NMR (400 MHz, Chloroform-d) δ 9.09 (s, 2H), 8.91 (s, 2H), 8.57 (dd, J (H−F)=9.8, 6.4 Hz, 2H), 8.46 (s, 2H), 8.06 (s, 2H), 7.75 (t, J (H−F)=7.5 Hz, 2H), 7.54 (d, J=1.4 Hz, 4H), 4.22 (d, J=5.5 Hz, 8H), 1.75 (d, J=6.6 Hz, 4H), 1.45-1.16 (m, 128H), 0.87 (td, J=6.9, 2.5 Hz, 24H).

The compound GS14 was also subjected to the other characterizations described above: the absorption spectrum, the optical energy gap (E_{g}°), the values of the energy levels HOMO (E_{HOMO}), LUMO (E_{LUMO}) and electrochemical band-gap (E_{gapEC}) have been acquired: the values obtained are reported in Table 2 and Table 3.

In Table 2 are reported, in order: the compound (Compound), the solvent used (Solvent), the value of the optical energy gap (E_{g}°), expressed in (eV), the maximum value of the lowest energy band in the absorption spectrum [λₘₐₓ (abs.)] expressed in (nm).

In Table 3 are reported, in order: the compound (Compound), the value of the HOMO (E_{HOMO}) energy level expressed in (eV), the value of the LUMO (E_{LUMO}) energy level expressed in (eV) and, finally the value of the electrochemical band-gap (E_{gapEC}) expressed in (eV).

FIG. 6 shows [the potential (E) measured in volts (V) vs ferrocene/ferrocinium (Fc/Fc⁺) is shown in the abscissa and the current density (i) measured in amperes (A) is shown in the ordinate)] the cyclic voltagram obtained by operating as described above.

### EXAMPLE 6

### Synthesis of Compound GS27: 2,2′-(((5,6-di(4-carbo(2-octyldodecyloxy)-phenoxy)benzo[c][1,2,5]thiadiazole-4,7-di-2-thienothiophene-5-diyl-bis(methanylidene)-bis-5,6-dichloro-3-oxo-2,3-dihydro-1H-indene-2,1-diylidene))dimalononitrile

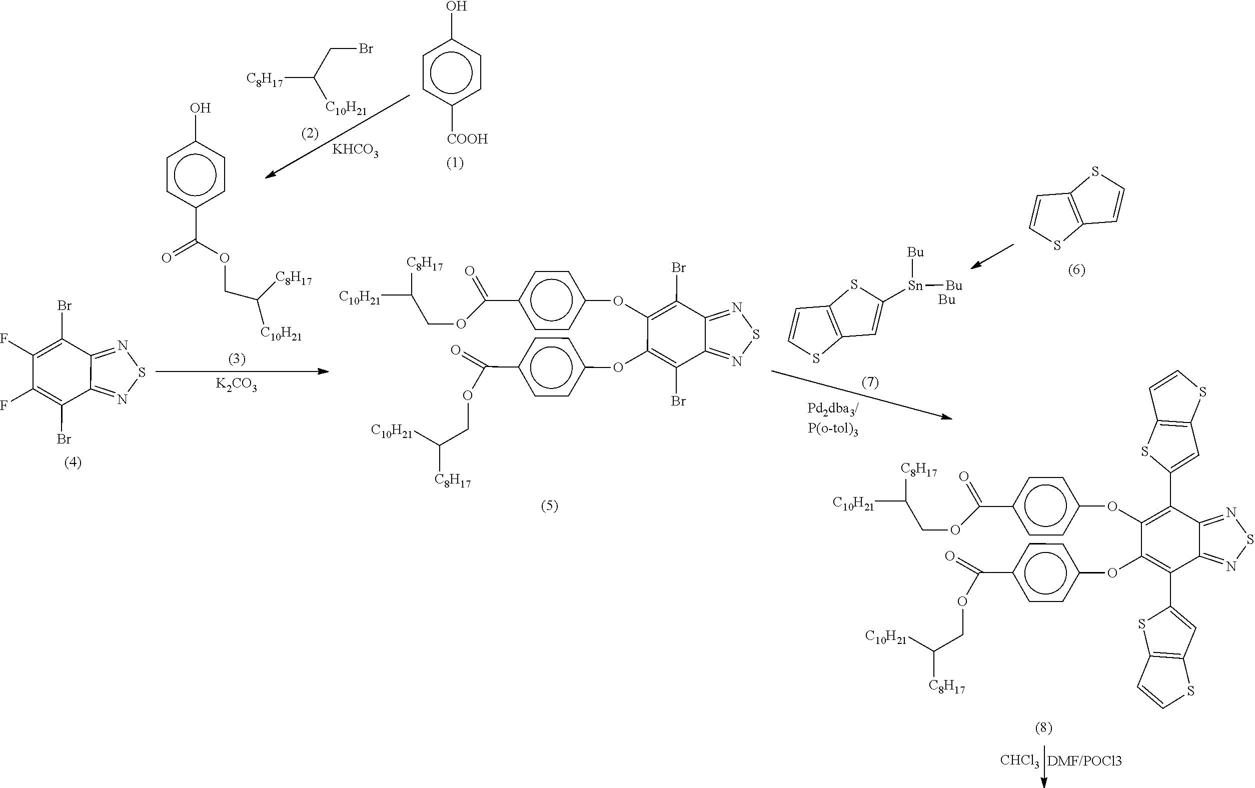

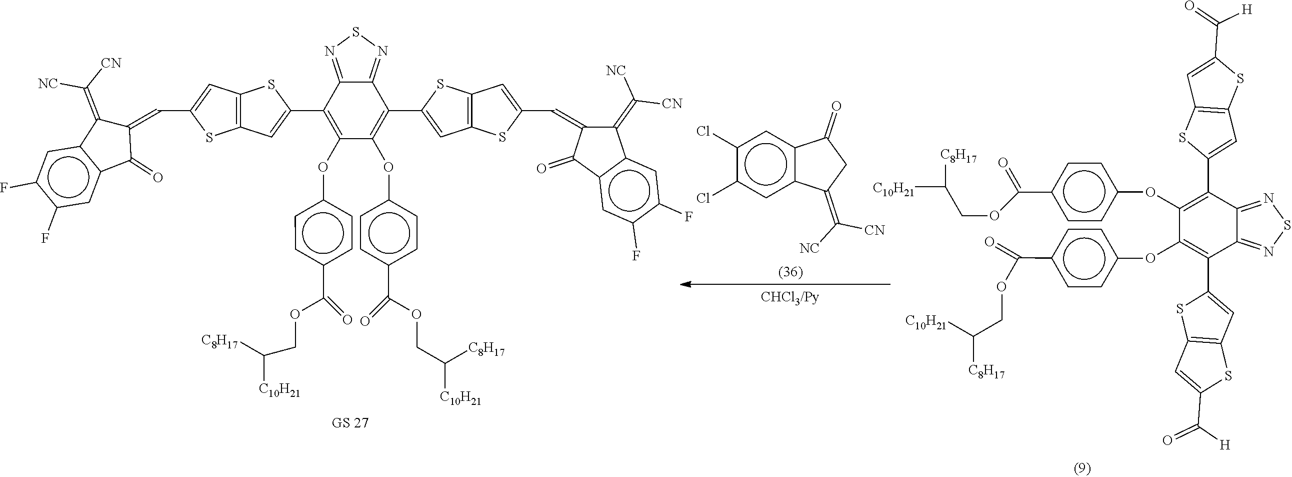

Anhydrous pyridine (Py) (Merck) (731 μl) was added to a solution of 4,7-di(2-(5-formylthienothienyl)-5,6-di(4-octyldodecylphenoxy)benzothiadiazole (9) (146, 5 mg; 0.11 mmol) obtained as described above, in anhydrous chloroform 5(Merck) (32 ml) in a 100 ml flask, equipped with magnetic stirring, thermometer and cooler, under an inert atmosphere (argon). After removing the air from the reaction environment by 3 vacuum/argon cycles, the flask was placed in an ethanol and dry ice bath and the reaction mixture was cooled to −10° C., and a solution, previously deaerated, using 3 vacuum/argon cycles, of 5,6-dichloro-3-10 (dicyanomethylidene)indan-1-one (36) (Sunatech) (118.3 mg; 0.45 mmol), in anhydrous chloroform (Merck) (10 ml) was added dropwise, in 10 minutes. After removing the air from the reaction environment by 3 vacuum/argon cycles, the temperature was allowed to rise spontaneously to 20° C. and the reaction mixture was maintained, at said temperature, under stiffing, for 10 minutes. Subsequently, the reaction mixture was heated to 65° C. and maintained, at said temperature, under stirring for 18 hours. Subsequently, the temperature was allowed to drop spontaneously to 20° C. and acetonitrile (Merck) (20 ml) was added: the reaction mixture was kept, at said temperature, under stirring, for 1 hour. Subsequently, most of the organic solvent was removed by distillation at reduced pressure and the remaining residue was taken up with chloroform (Merck) (10 ml): the mixture obtained was added, dropwise, to acetonitrile (Merck) (20 ml). The precipitate obtained was isolated by filtration, washed with acetonitrile (Merck) (5×10 ml), ethanol (Merck) (1×3 ml) and, finally, with ethyl ether (Merck) (1×3 ml) obtaining 160.0 mg (0.082 mmol) of the compound GS27 (yield=75%).

The compound GS27 was subjected to the characterizations listed above: the absorption spectrum, the optical energy gap (E_{g}°), the values of the energy levels HOMO (E_{HOMO}), LUMO (E_{LUMO}) and electrochemical band-gap (E_{gapEC}) were acquired: the values obtained are reported in Table 2 and Table 3.

In Table 2 are reported, in order: the compound (Compound), the solvent used (Solvent), the value of the optical energy gap (E_{g}°), expressed in (eV), the maximum value of the lowest energy band in the absorption spectrum [λₘₐₓ (abs.)] expressed in (nm).

In Table 3 are reported, in order: the compound (Compound), the value of the HOMO (E_{HOMO}) energy level expressed in (eV), the value of the LUMO (E_{LUMO}) energy level expressed in (eV) and, finally the value of the electrochemical band-gap (E_{gapEC}) expressed in (eV).

FIG. 7 shows [the potential (E) measured in volts (V) vs ferrocene/ferrocinium (Fc/Fc⁺) is shown on the abscissa and the current density (i) measured in amperes (A) is shown on the ordinate)] the cyclic voltagram obtained by operating as described above.

### EXAMPLE 7

### Synthesis of Compound GS034: 2,2′-(((5,6-di(4-carbo(2-hexyldecyloxy)-phenoxy)benzo[c][1,2,5]thiadiazole-4,7-di-2-(6-octylthienothiophene-5-diyl)]-bis(methanylidene)-bis-5,6-difluoro-3-oxo-2,3-dihydro-1H-indene-2,1-diylidene))dimalononitrile

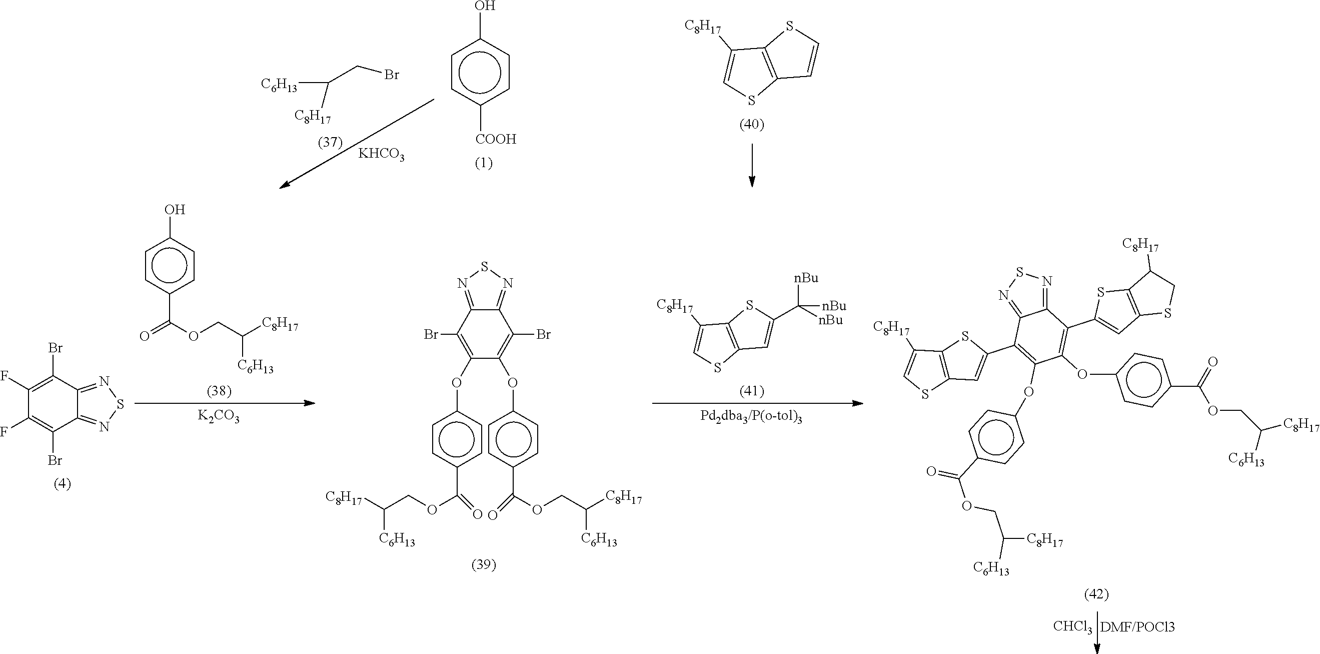

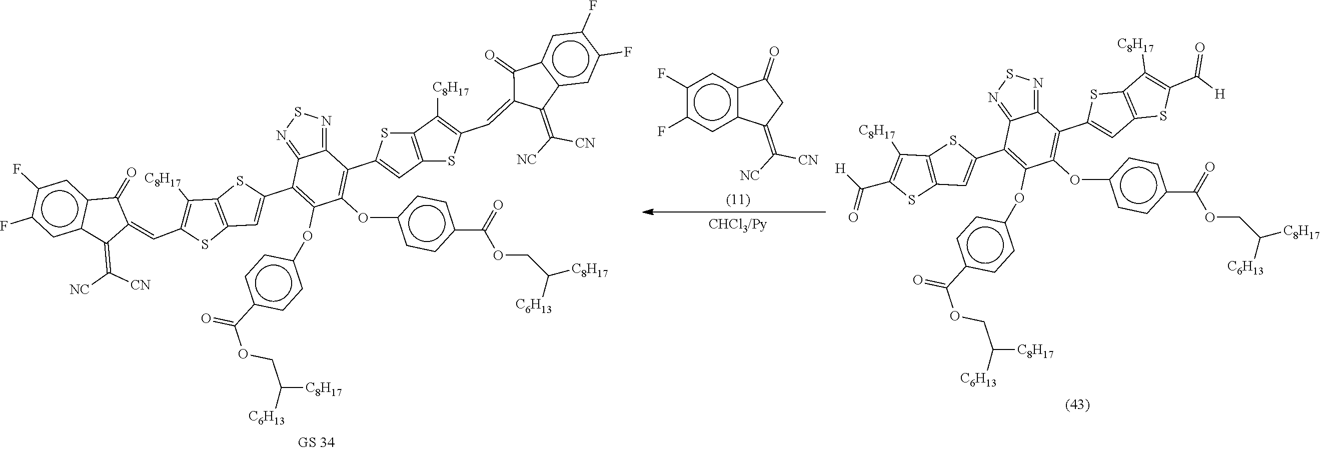

### Synthesis of 2-hexyldecyl 4-hydroxybenzoate (38)

The following were loaded into a 50 ml microwaveable vial: 4-hydroxybenzoic acid (1) (Merck) (1.5 g; 10.86 mmol), 2-hexyldecylbromide (37) (Merck) (3.3 g; 10.86 mmol), anhydrous N,N-dimethylformamide (Merck) (30 ml), potassium bicarbonate (Merck) (KHCO₃) (1.08 g; 10.86 mmol) and potassium iodide (Merck) (180.6 mg; 1.08 mmol): after insufflation with argon, the vial was placed in the microwave (Discover SP-D—CEM Corp.). After 1 hour, at 80° C., under stirring (medium stirring), the reaction mixture was poured into distilled water (50 ml) and extracted with ethyl ether (Merck) (3×30 ml). The organic phase (obtained by combining the three organic phases) was washed to neutral with distilled water (3×20 ml) and dried over sodium sulphate (Merck). The solvent was removed by distillation at reduced pressure and the residue obtained was purified by elution on a silica gel chromatographic column (eluent: n-heptane (Merck)/eluent 1 in gradient from 95/5 to 90/10 to 80/20, wherein the eluent 1 consist of a mixture of dichloromethane (Merck):ethyl acetate (Merck) in the ratio 1:1 v/v), obtaining 2.5 g (7.1 moles) of 2-hexyldecyl 4-hydroxybenzoate (38) (yield=66%).

### Synthesis of 4,7-dibromo-5,6-di(4-carbo(2-hexyldecyloxy)phenoxy)benzo-thiadiazole (39)

2-hexyldecyl 4-hydroxybenzoate (38) (1.5 g; 4.1 mmol) obtained as described above and potassium carbonate (K₂CO₃) (Merck) (571.3 mg; 4.1 mmol) were added to a solution of 4,7-dibromo-5,6-difluorobenzothiadiazole (4) (Merck) (637.0 mg; 1.9 mmol) in N,N-dimethylformamide (anhydrous) (Merck) (12 ml) in a 100 ml flask, equipped with magnetic stirring, thermometer and cooler, in an inert atmosphere: the mixture obtained was heated to 82° C. and kept, under stirring, at said temperature, for 12 hours. Subsequently, the reaction mixture was poured into distilled water (50 ml) and was extracted with ethyl ether (Merck) (3×30 ml). The organic phase (obtained by combining the three organic phases) was washed to neutral with distilled water (3×30 ml) and dried over sodium sulphate (Merck). The solvent was removed by distillation at reduced pressure and the residue obtained was purified by elution on a silica gel chromatographic column (eluent: n-heptane (Merck)/dichloromethane (Merck) in a gradient from 95/5 to 90/10 to 80/20) obtaining 1.8 g (1.8 mmoles) of 4,7-dibromo-5,6-di(4-carbo(2-hexyldecyloxy)phenoxy)benzothiadiazole (39) (yield=96%).

### Synthesis of 2-tri-n-butylstannyl-6-octylthienothiophene (41)

n-Butyllithium [1.6 M solution in hexane (Merck)](2.8 ml; 4.49 mmol) was added dropwise to a 0.12 M solution of 3-octylthienothiophene (40) (Merck) (1.03 g; 4.08 mmol) in anhydrous tetrahydrofuran (Merck) (50 ml) in a 100 ml flask, equipped with magnetic stirring, thermometer and cooler, placed in a dry ice bath at −78° C., in an inert atmosphere (argon): the reaction mixture obtained was kept under stirring, and the temperature was allowed to rise spontaneously to −50° C., in 3 hours. Subsequently, after bringing the temperature back to −78° C., tri-n-butylstannylchloride (Merck) (1.6 g; 1.32 ml; 4.9 mmol) was added dropwise: after 15 minutes the flask was removed from the dry ice bath, the temperature was allowed to rise spontaneously to 20° C. and the reaction mixture was kept, at said temperature, under stirring, for 12 hours. Subsequently, after adding a saturated aqueous solution of sodium bicarbonate (Merck) (20 ml), the reaction mixture was extracted with diethyl ether (Merck) (3×25 ml). The organic phase (obtained by combining the three organic phases) was washed with a saturated aqueous solution of sodium bicarbonate (Merck) (1×30 ml) and dried over sodium sulphate (Merck). The solvent was removed by distillation under reduced pressure obtaining 2-tri-n-butylstannyl-6-octylthienothiophene (41) which is used as such in the subsequent reaction.

### Synthesis of 4,7-di[2-(6-octylthienothienyl)]-5,6-di(4-carbo(2-hexyldecyloxy)phenoxy)benzo-thiadiazole (42)

4,7-dibromo-5,6-di(4-carbo(2-hexyldecyloxy)phenoxy)benzothia-diazole (39) (1.7 g; 1.7 mmol) obtained as described above was added to a solution of 2-tri-n-butylstannyl-6-octylthienothiophene (41) (4.08 mmol) obtained as described above in anhydrous toluene (Merck) (38 ml) in a 100 ml flask, equipped with magnetic stirring, thermometer and cooler, under inert atmosphere. After removing the air present through 3 vacuum/nitrogen cycles, tris-dibenzylideneacetone dipalladium (Pd₂dba₃) (Merck) (40.6 mg; 0.044 mmol) and tris-o-tolylphosphine [P(o-tol)₃](Merck) (53.6 mg; 0.18 mmoles) were added: the mixture obtained was heated to 108° C. and kept under stirring at said temperature for 12 hours. Subsequently, after adding distilled water (50 ml), the reaction mixture was extracted with ethyl acetate (Merck) (3×50 ml). The organic phase (obtained by combining the three organic phases) was washed to neutral with distilled water (3×50 ml) and dried over sodium sulphate (Merck). After removing the solvent by distillation under reduced pressure, the residue obtained was purified by elution on a silica gel chromatographic column (eluent: n-heptane (Merck)/eluent 1 in the ratio 9/1 v/v, wherein the eluent 1 consists of a mixture of dichloromethane (Merck)/ethyl acetate (Merck) in the ratio 1/1 v/v), obtaining 1.7 g (1.25 mmol) of 4,7-di[2-(6-octylthienothienyl)]-5,6-di(4-carbo(2-hexyldecyloxy)phenoxy)benzo-thiadiazole (42) (yield=70%).

### Synthesis of 4,7-di(2-(5-formyl-6-octylthienothienyl)-5,6-di(4-carbo(2-hexyldecyloxy)phenoxy)benzothiadiazole (43)

N,N-dimethylformamide (DMF) (Merck) (3.9 ml) and, dropwise, phosphorus oxychloride (POCl₃) (Merck) (3.1 ml; 5.08 g; 33.1 mmol) were added to a solution of 4,7-di[2-(6-octylthienothienyl)]-5,6-di(4-carbo(2-hexyldecyloxy)phenoxy)benzothiadiazole (42) (1.7 g; 1.25 mmol) obtained as described above in anhydrous chloroform (CHCl₃) (Merck) (46.2 ml) in a 100 ml flask, equipped with magnetic stirring, thermometer and cooler, under an inert atmosphere, at 0° C.: the reaction mixture was placed under stirring and, after 30 minutes, it was heated to 69° C. and kept under stirring at said temperature for 48 hours. Subsequently, after adding a 10% solution of potassium acetate in water (20 ml), the reaction mixture was kept, under stirring, at 69° C., for 1 hour and subsequently extracted with ethyl acetate (Merck) (3×30 ml). The organic phase (obtained by combining the three organic phases) was washed to neutral with distilled water (3×30 ml) and dried over sodium sulphate (Merck). After removing the solvent by distillation under reduced pressure, the residue obtained was purified by elution on a silica gel chromatographic column (eluent n-heptane (Merck)/eluent 1 in gradient from 95/5 to 9/1 to 85/15 to 8/2, wherein the eluent 1 consists of a mixture of dichloromethane (Merck)/ethyl acetate (Merck) in the ratio 1/1 v/v), obtaining 910.0 mg (0.7 mmoles) of 4,7-di(2-(5-formyl-6-octylthienothienyl)-5,6-di(4-carbo(2-hexyldecyloxy)phenoxy)-benzothiadiazole (43) (yield 83.3%).

### Synthesis of compound GS034: 2,2′-(((5,6-di(4-carbo(2-hexyldecyloxy)-phenoxy)benzo[c][1,2,5]thiadiazole-4,7-di-2-(6-octylthienothiophene-5-diyl)]-bis(methanylidene)-bis-5,6-difluoro-3-oxo-2,3-dihydro-1H-indene-2,1-diylidene))dimalononitrile

Anhydrous pyridine (Merck) (3.87 ml) was added to a solution of 4,7-di(2-(5-formyl-6-octylthienothienyl)-5,6-di(4-carbo(2-hexyldecyloxy)phenoxy)-benzothiadiazole (43) (918.0 mg; 0.65 mmol) obtained as described above, in anhydrous chloroform (CHCl₃) (Merck) (180 ml) in a 100 ml flask, equipped with magnetic stirring, thermometer and cooler, in an inert atmosphere (argon). After removing the air from the reaction environment, by means of 3 vacuum/argon cycles, the flask was placed in an ethanol and dry ice bath, the reaction mixture was cooled to −10° C., and added, dropwise, in 10 minutes, a solution, previously deaerated, by means of 3 vacuum/argon cycles, of 5,6-difluoro-3-(dicyanomethylidene)indan-1-one (11) (Merck) (597.0 mg; 2.6 mmol), in anhydrous chloroform (Merck) (30 ml). After removing the air from the reaction environment by means of 3 vacuum/argon cycles, the temperature was allowed to rise spontaneously to 20° C. and the reaction mixture was kept at said temperature, under stirring, for 10 minutes. Subsequently, the reaction mixture was heated to 65° C. and maintained, at said temperature, under stirring for 18 hours. Subsequently, the temperature was allowed to drop spontaneously to 20° C. and acetonitrile (Merck) (20 ml) was added: the reaction mixture was kept, at said temperature, under stirring, for 1 hour. Subsequently, most of the organic solvent was removed by distillation at reduced pressure and the remaining residue was taken up with chloroform (Merck) (10 ml): the mixture obtained was added, dropwise, to acetonitrile (Merck) (20 ml). The precipitate obtained was isolated by filtration, washed with acetonitrile (Merck) (5×10 ml), ethanol (Merck) (1×3 ml) and, finally, with ethyl ether (Merck) (1×10 ml) obtaining 850.0 mg (0.46 mmol) of compound GS034 (yield=71%).

The compound GS034 was subjected to the characterizations reported above: the absorption spectrum, the optical energy gap (E_{g}°), the values of the energy levels HOMO (E_{HOMO}), LUMO (E_{LUMO}) and the electrochemical band-gap (E_{gapEC}) have been acquired: the values obtained are reported in Table 2 and Table 3.

In Table 2 are reported, in order: the compound (Compound), the solvent used (Solvent), the value of the optical energy gap (E_{g}°), expressed in (eV), the maximum value of the lowest energy band in the absorption spectrum [λₘₐₓ (abs.)] expressed in (nm).

In Table 3 are reported, in order: the compound (Compound), the value of the HOMO (E_{HOMO}) energy level expressed in (eV), the value of the LUMO (E_{LUMO}) energy level expressed in (eV) and, finally the value of the electrochemical band-gap (E_{gapEC}) expressed in (eV).

FIG. 8 shows [the potential (E) measured in volts (V) vs ferrocene/ferrocinium (Fc/Fc⁺) is reported on the abscissa and the current density (i) measured in amperes (A) is reported on the ordinate)] the cyclic voltagram obtained by operating as described above.

TABLE 2

| | | E_{g}° | λₘₐₓ |
|---|---|---|---|
| Compound | Solvent | (eV) | (nm) |
| GS7 | dichlorobenzene | 1.80 | 639 |
| GS8 | dichlorobenzene | 1.77 | 648 |
| GS12 | o-xylene | 1.80 | 640 |
| GS13 | o-xylene | 1.82 | 638 |
| GS14 | o-xylene | 1.82 | 635 |
| GS27 | o-xylene | 1.86 | 642 |
| GS034 | o-xylene | 1.79 | 647 |

TABLE 3

| | E_{HOMO} | E_{LUMO} | E_{gapEC} |
|---|---|---|---|
| Compound | (eV) | (eV) | (eV) |
| GS7 | −5.93 | −3.94 | 1.99 |
| GS8 | −5.99 | −4.05 | 1.94 |
| GS12 | −5.92 | −4.02 | 1.90 |
| GS13 | −5.95 | −4.02 | 1.93 |
| GS14 | −5.99 | −3.95 | 2.04 |
| GS27 | −5.94 | −4.06 | 1.88 |
| GS034 | −6.01 | −3.91 | 2.10 |

1. A diaryloxybenzoheterodiazole compound disubstituted with thienothiophene groups having general formula (I): 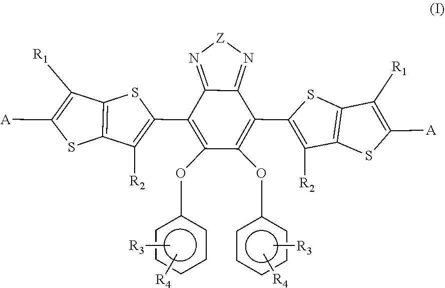
wherein:
Z represents a sulfur atom, an oxygen atom, a selenium atom; or a NR₅ group wherein R₅ is selected from C₁-C₃₀, preferably C₁-C₂₀, linear or branched alkyl groups, or from aryl groups optionally substituted;
R₁ and R₂, identical to or different from each other, represent a hydrogen atom; or are selected from C₁-C₃₀, preferably C₁-C₂₀, linear or branched alkyl groups, optionally containing heteroatoms, cycloalkyl groups optionally substituted, aryl groups optionally substituted, C₁-C₃₀, preferably C₁-C₂₀, linear or branched alkoxyl groups, optionally substituted, —COOR₆ groups wherein R₆ is selected from C₁-C₃₀, preferably C₁-C₂₀, linear or branched alkyl groups, or is a cyano group;
R₃ and R₄, identical to or different from each other, represent a hydrogen atom; or are selected from C₁-C₃₀, preferably C₁-C₂₀, linear or branched alkyl groups, optionally containing heteroatoms, C₁-C₃₀, preferably C₁-C₂₀, linear or branched alkoxyl groups, optionally substituted, —COOR₇ or —OCOR₇ groups wherein R₇ is selected from C₁-C₃₀, preferably C₁-C₂₀, linear or branched alkyl groups, optionally containing heteroatoms, thioether groups —R₈—S—R₉ wherein R₈ and R₉, identical to or different from each other, are selected from C₁-C₃₀, preferably C₁-C₂₀, linear or branched alkyl groups, optionally containing heteroatoms; and
A represents an electron acceptor group having general formula (II): 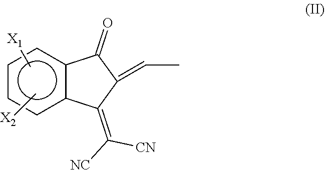
wherein X₁ and X₂, identical to or different from each other, represent a hydrogen atom, or a halogen atom such as chlorine, fluorine, bromine, preferably chlorine, fluorine; or are selected from C₁-C₃₀, preferably C₁-C₂₀, linear or branched alkyl groups, optionally containing heteroatoms, C₁-C₃₀, preferably C₁-C₂₀, linear or branched alkoxyl groups, optionally substituted; or are selected from —COOR₁₀ ester groups wherein R₁₀ is selected from C₁-C₃₀, preferably C₁-C₂₀, linear or branched alkyl groups, optionally containing heteroatoms, C₁-C₃₀, preferably C₁-C₂₀, linear or branched alkoxyl groups, optionally substituted.

2. The diaryloxybenzoheterodiazole compound disubstituted with thienothiophene groups according to claim 1, wherein in said general formula (I):
Z represents a sulfur atom;
R₁ and R₂, identical to each other, represent a hydrogen atom; or R₁ and R₂, different from each other, represent a hydrogen atom or a C₁-C₃₀ alkyl group, preferably R₁ represents n-octyl and R₂ represents a hydrogen atom;
R₃ and R₄, identical to or different from each other, represent a hydrogen atom, or represent a —COOR₇ group wherein R₇ represents a C₁-C₃₀ alkyl group, preferably 2-octyldodecyl, 2-hexyldecyl, 2-butyloctyl, said —COOR₇ group being in position 2, or in position 3, or in position 4, or in position 3,5 of the phenyl; and
A represents an electron acceptor group having general formula (II) wherein X₁ and X₂, identical to each other, represent a hydrogen atom, a fluorine atom, or a chlorine atom.

3. An organic photovoltaic device (or solar device) selected from organic, binary, ternary, quaternary photovoltaic cells (or solar cells), having both simple and tandem architecture, organic photovoltaic modules (or solar modules), both on rigid support and on a flexible support, comprising at least one diaryloxybenzoheterodiazole compound disubstituted with thienothiophene groups having general formula (I) recited in claim 1, preferably an organic, binary, ternary, quaternary photovoltaic cell (or solar cell), having both simple and tandem architecture.

4. A binary, ternary, quaternary organic photovoltaic cell (or solar cell), having both simple and tandem architecture, comprising:
at least one rigid or flexible support;
an anode;
at least one layer of photoactive material; and
a cathode;
wherein said layer of photoactive material comprises at least one photoactive organic polymer as electron donor compound, at least one diaryloxybenzoheterodiazole compound disubstituted with thienothiophene groups having general formula (I) recited in claim 1 as electron acceptor compound.

5. The organic photovoltaic cell (or solar cell) binary, ternary, quaternary, having both simple and tandem architecture, according to claim 4, wherein said photoactive organic polymer is selected from:
(a) polythiophenes such as regioregular poly (3-hexylthiophene) (P3HT), poly (3-octylthiophene), poly (3,4-ethylenedioxythiophene), or mixtures thereof;
(b) alternating or statistical conjugated copolymers comprising:
at least one benzotriazole (B) unit having general formula (Ia) or (Ib): 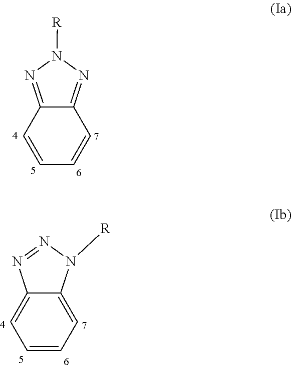
wherein R group is selected from alkyl groups, aryl groups, acyl groups, thioacyl groups, said alkyl, aryl, acyl and thioacyl groups being optionally substituted;
at least one conjugated structural unit (A), wherein each unit (B) is connected to at least one unit (A) in any one of the positions 4, 5, 6, or 7, preferably in the positions 4 or 7;
(c) alternating conjugated copolymers comprising benzothiadiazole units such as PCDTBT {poly[N-9″-heptadecanyl-2,7-carbazole-alt-5,5-(4′,7′-di-2-thienyl-2′,1′,3′-benzothiadiazole]}, PCPDTBT {poly[2,6-(4,4-bis-(2-ethyl-hexyl)-4H-cyclopenta[2,1-b;3,4-b′]-dithiophene)-alt-4,7-(2,1,3-benzothia-diazole)]};
(d) alternating conjugated copolymers comprising thieno[3,4-b]pyrazidine units;
(e) alternating conjugated copolymers comprising quinoxaline units;
(f) alternating conjugated copolymers comprising silolics monomer units such as 9,9-dialkyl-9-silafluorene copolymers;
(g) alternating conjugated copolymers comprising condensed thiophene units such as copolymers of thieno[3,4-b]thiophene and benzo[1,2-b:4,5-b′]dithiophene;
(h) alternating conjugated copolymers comprising benzothiadiazole or naphthothiadiazole units substituted with at least one fluorine atom and thiophene units substituted with at least one fluorine atom such as PffBT4T-2OD {poly[(5,6-difluoro-2,1,3-benzothiadiazole-4,7-diyl)-alt-(3,3′″-di(2-octyldodecyl)-2,2′,5′,2″,5″,2′″-quaterthiophen-5,5′″-diyl)]}, PBTff4T-2OD {poly[(2,1,3-benzothiadiazole-4,7-diyl)-alt-(4′,3″-difluoro-3,3′″-di(2-octyldodecyl)-2,2′;5′,2″;5″,2′″-quaterthiophene-5,5′″-diyl)]}, PNT4T-2OD {poly(naphtho[1,2-c:5,-c′]bis[1,2,5]thiadiazole-5,10-diyl)-alt-(3,3′″-di(2-octyldodecyl)-2,2′;5′,2″;5″,2′″-quaterthiophene-5,5′″-diyl)]};
(i) conjugated copolymers comprising thieno[3,4-c]pyrrole-4,6-dione units such as PBDTTPD {poly[[5-(2-ethylhexyl)-5,6-dihydro-4,6-dioxo-4H-thieno[3,4-c]pyrrole-1,3-diyl][4,8-bis[(2-ethylhexyl)oxy]benzo[1,2-b:4,5-b′]dithiophene-2,6-diyl]};
(l) conjugated copolymers comprising thienothiophenic units such as PTB7 {poly({4,8-bis[(2-ethylhexyl)oxy]benzo[1,2-b:4,5-b′]dithiophene-2,6-diyl}{3-fluoro-2-[(2-ethylhexyl)carbonyl]thieno[3,4-b]thiophenediyl}}, PBDB-T polymer poly[[4,8-bis[5-(2-ethylhexyl)-2-thienyl]benzo[1,2-b:4,5-b′]-dithiophene-2,6-diyl]-2,5-thiophenediyl[5,7-bis(2-ethylhexyl)-4,8-dioxo-4H,8H-benzo[1,2-c:4,5-c′]dithiophene-1,3-diyl]-2,5-thiophenediyl]; and
(m) polymers comprising a derivative of indacen-4-one having general formula (III), (IV) or (V): 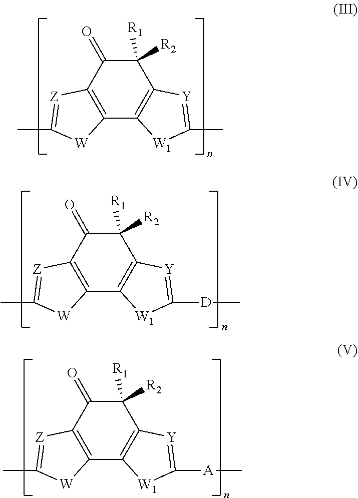
wherein:
W and W₁, identical to or different from each other, preferably identical to each other, represent an oxygen atom; a sulfur atom; an N—R₃ group wherein R₃ represents a hydrogen atom, or is selected from C₁-C₂₀, preferably C₂-C₁₀, linear or branched alkyl groups;
Z and Y, identical to or different from each other, preferably identical to each other, represent a nitrogen atom; or a CR₄ group wherein R₄ represents a hydrogen atom, or is selected from C₁-C₂₀, preferably C₂-C₁₀, linear or branched alkyl groups, cycloalkyl groups optionally substituted, aryl groups optionally substituted, heteroaryl groups optionally substituted, C₁-C₂₀, preferably C₂-C₁₀, linear or branched alkoxyl groups, polyethyleneoxyl groups R₅—O—[CH₂—CH₂—O]ₙ— wherein R₅ is selected from C₁-C₂₀, preferably C₂-C₁₀, linear or branched alkyl groups, and n is an integer ranging from 1 to 4, —R₆—OR₇ groups wherein R₆ is selected from C₁-C₂₀, preferably C₂-C₁₀, linear or branched alkylene groups and R₇ represents a hydrogen atom or is selected from C₁-C₂₀, preferably C₂-C₁₀, linear or branched alkyl groups, or is selected from polyethyleneoxyl groups R₅—[—OCH₂—CH₂—O]ₙ— wherein R₅ has the same meanings reported above and n is an integer ranging from 1 to 4, —COR₈ groups wherein R₈ is selected from C₁-C₂₀, preferably C₂-C₁₀, linear or branched alkyl groups, —COOR₉ groups wherein R₉ is selected from C₁-C₂₀, preferably C₂-C₁₀, linear or branched alkyl groups; or represent a —CHO group, or a cyano group (—CN);
R₁ and R₂, identical to or different from each other, preferably identical to each other, are selected from C₁-C₂₀, preferably C₂-C₁₀, linear or branched alkyl groups; cycloalkyl groups optionally substituted; aryl groups optionally substituted; heteroaryl groups optionally substituted; C₁-C₂₀, preferably C₂-C₁₀, linear or branched alkoxyl groups; polyethyleneoxyl groups R₅—O—[CH₂—CH₂—O]ₙ— wherein R₅ has the same meanings reported above and n is an integer ranging from 1 to 4; —R₆—OR₇ groups wherein R₆ and R₇ have the same meanings reported above; —COR₈ groups wherein R₈ has the same meanings reported above; —COOR₉ groups wherein R₉ has the same meanings reported above; or represent a —CHO group, or a cyano group (—CN);
D represents an electron-donor group;
A represents an electron acceptor group; and
n is an integer ranging from 10 to 500;
(n) polymers comprising antradithiophene derivatives having general formula (X): 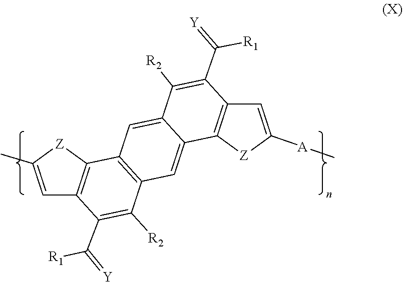
wherein:
Z, identical to or different from each other, preferably identical to each other, represent a sulfur atom, an oxygen atom, a selenium atom;
Y, identical to or different from each other, preferably identical to each other, represent a sulfur atom, an oxygen atom, a selenium atom;
R₁, identical to or different from each other, preferably identical to each other, are selected from amino groups —N—R₃R₄ wherein R₃ represents a hydrogen atom, or is selected from C₁-C₂₀, preferably C₂-C₁₀, linear or branched alkyl groups, or is selected from cycloalkyl groups optionally substituted and R₄ is selected from C₁-C₂₀, preferably C₂-C₁₀, linear or branched alkyl groups, or is selected from cycloalkyl groups optionally substituted; or are selected from C₁-C₃₀, preferably C₂-C₂₀, linear or branched alkoxy groups; or are selected from polyethyleneoxyl groups R₅—O—[CH₂—CH₂—O]ₙ— wherein R₅ is selected from C₁-C₂₀, preferably C₂-C₁₀, linear or branched alkyl groups, and n is an integer ranging from 1 to 4; or are selected from —R₆—OR₇ groups wherein R₆ is selected from C₁-C₂₀, preferably C₂-C₁₀, linear or branched alkylene groups and R₇ represents a hydrogen atom, or is selected from C₁-C₂₀, preferably C₂-C₁₀, linear or branched alkyl groups, or is selected from polyethyleneoxyl groups R₅—[—OCH₂—CH₂-]ₙ— wherein R₅ has the same meanings reported above and n is an integer ranging from 1 to 4; or are selected from thiol groups —S—R₅ wherein R₅ is selected from C₁-C₂₀, preferably C₂-C₁₀, linear or branched alkyl groups;
R₂, identical to or different from each other, preferably identical to each other, represent a hydrogen atom; or are selected from C₁-C₂₀, preferably C₂-C₁₀, linear or branched alkyl groups; or are selected from —COR₉ groups wherein R₉ is selected from C₁-C₂₀, preferably C₂-C₁₀, linear or branched alkyl groups; or are selected from —COOR₁₀ groups wherein R₁₀ is selected from C₁-C₂₀, preferably C₂-C₁₀, linear or branched alkyl groups; or are selected from aryl groups optionally substituted; or are selected from heteroaryl groups optionally substituted;
A represents an electron-acceptor group; and
n is an integer ranging from 10 to 500.

6. The ternary organic photovoltaic cell (or solar cell), having both simple and tandem architecture, according to claim 4, wherein the photoactive layer comprises:
two photoactive organic polymers selected from those according to claim 4 and one diaryloxybenzoheterodiazole compound disubstituted with thienothiophene groups having general formula (I); or
one photoactive organic polymer selected from those according to claim 4 and two diaryloxybenzoheterodiazole compounds disubstituted with thienothiophene groups having general formula (I); or
one photoactive organic polymer selected from those according to claim 4, one diaryloxybenzoheterodiazole compound disubstituted with thienothiophene groups having general formula (I) and one fullerene derivative such as PC61BM (6,6-phenyl-C₆₁-methyl butyric ester) or the PC71BM (6,6-phenyl-C₇₁-methyl butyric ester); or
one photoactive organic polymer selected from those according to claim 4, one diaryloxybenzoheterodiazole compound disubstituted with thienothiophene groups having general formula (I) and one non-fullerene compound selected from non-fullerene compounds, optionally polymeric, such as compounds based on perylene-diimides or naphthalene-diimides and fused aromatic rings; indacenothiophenes with electron-poor terminal groups; compounds having an aromatic core capable of symmetrically rotating such as derivatives of corannulene or truxenone.

7. The guaternary organic photovoltaic cell (or solar cell), having both simple and tandem architecture, according to claim 4, wherein the photoactive layer comprises:
two photoactive organic polymers selected from those according to claim 4, and two diaryloxybenzoheterodiazole compounds disubstituted with thienothiophene groups having general formula (I); or
two photoactive organic polymers selected from those according to claim 4, one diaryloxybenzoheterodiazole compound disubstituted with thienothiophene groups having general formula (I) and one fullerene derivative such as PC61BM (6,6-phenyl-C₆₁-methyl butyric ester) or PC71BM (6,6-phenyl-C₇₁-methyl butyric ester); or
two photoactive organic polymers 4, one diaryloxybenzoheterodiazole compound disubstituted with thienothiophene groups having general formula (I) and one non-fullerene compound selected from non-fullerene compounds, possibly polymeric, such as compounds based on perylene-diimides or naphthalene-diimides and fused aromatic rings; indacenothiophenes with electron-poor terminal groups; compounds having an aromatic core capable of symmetrically rotating such as derivatives of corannulene or truxenone.

8. Perovskite-based photovoltaic cell (or solar cell) wherein the electron-carrying material-based layer (Electron Transport Layer—ETL) comprises at least one diaryloxybenzoheterodiazole compound disubstituted with thienothiophene groups having general formula (I) according to claim 1.

9. Organic thin film transistors (OTFTs), or organic field effect transistors (OFETs) comprising at least one diaryloxybenzoheterodiazole compound disubstituted with thienothiophene groups having general formula (I) according to claim 1.
